(19) United States
(12) Patent Application Publication
(10) Pub. No.: US 2005/0070719 A1
(43) Pub. Date: Mar. 31, 2005

(54) Inhibitors of dipeptidyl peptidase iv

(76) Inventors: Sergei Belyakov, Baltimore, MD (US); Gregory S Hamilton, Catonsville, MD (US); David Chadwick Hurst, Santee, CA (US); Vincent J Kalish, Annapolis, MD (US); Weixing Li, Ellicot City, MD (US); Eric Yanjun Wang, San Diego, CA (US); Douglas Wilkinson, Baltimore, MD (US); Yong-Qian Wu, Columbia, MD (US); Weizheng Xu, Ellicot City, MD (US)

Correspondence Address: HELLER EHRMAN WHITE & MCAULIFFE LLP, 1666 K STREET,NW, SUITE 300, WASHINGTON, DC, 20006, US

(21) Appl. No.: 10/499,675

(22) PCT Filed: Dec. 26, 2002
(86) PCT No.: PCT/US02/41469

Related U.S. Application Data
(60) Provisional application No. 60/342,092, filed on Dec. 26, 2001.
(60) Provisional application No. 60/407,947, filed on Sep. 5, 2002.

(51) Int. Cl. C07D 277/04; C07D 233/60

(52) U.S. Cl. 548/200; 548/215; 548/530; 548/334.1

(57) ABSTRACT

Novel inhibitors of dipeptidyl peptidase IV (DPP IV), pharmaceutical compositions comprising therapeutically effective amounts of novel inhibitors of DPP IV, and novel methods of treating medical conditions are provided. The novel inhibitors of DPP IV described herein are useful in the treatment of neurological disorders, diabetes, inflammatory disorders such as arthritis, obesity, osteoporosis, and of such other enumerated conditions as can be treated with inhibitors of DPP IV.

This application claims priority to U.S. Ser. No. 60/342,092, filed Dec. 26, 2001, and U.S. Ser. No. 60/407,947, filed Sep. 5, 2002, the entirety of which are hereby incorporated by reference.

The present invention relates to new and improved inhibitors of Dipeptidyl Peptidase IV, and new and improved treatment methods and related uses. The inhibitors according to the invention are useful for treating a wide variety of diseases and other abnormal conditions, including diseases impacting the central nervous system.

Dipeptidyl peptidase IV (DPP IV, EC 3.4.14.5) is a membrane-anchored aminopeptidase involved in the release of N-terminal dipeptides from proteins and other types or forms of peptides. The enzyme is a type II membrane serine peptidase, and has a substrate preference for proteins or peptides which carry a proline at the penultimate position of their N-termini. Since the peptide bonds before and after proline residues are known to be relatively resistant to cleavage by common proteases, it has been speculated that the presence of proline at the penultimate position of the peptide chain—a feature shared by a number of immunopeptides, neuropeptides, and peptide hormones—protects such peptides from degradation by unspecific exopeptidases. A physiological role for DPP IV would be in the activation, inactivation, or degradation of its substrates through the specific release of a proline-containing dipeptide from the N-terminal region of the substrate peptide.

DPP IV has been found in the kidney, epithelial cells, endothelial cells, small intestine, prostate, brain, placenta, and liver. In T-cells, it has been shown to be identical to the memory cell surface antigen CD26. Other proteins which display DPP IV-like activity include fibroblast-activation protein (FAP), an inducible type-II cell-surface glycoprotein selectively expressed by reactive stromal fibroblasts of epithelial cancers and healing wounds [Niedermeyer, et al., Eur. J. Biochem. 1998 254 (1998):650-4] and attractin/mahogany protein, which exists in membrane-bound and secreted forms and is implicated in control of pigmentation, energy metabolism, and CNS myelination [Tang et al., Proc. Natl. Acad. Sci. U.S.A. 97 (2000) 6025-30.].

DPP IV activity has also been found in serum, urine, seminal plasma, and amniotic fluid. It has been speculated that this soluble DPP IV activity can be attributed to cleavage of the membrane-bound form of DPP IV and release of its catalytic portion into the bloodstream [Augustyns, K., et al., Current Medicinal Chemistry, 6 (1999) 311-327]. Additionally, a distinct form of DPP IV, which appears to be a breakdown product of the T-cell surface antigen DPPT-L, has been described in human plasma. [Duke-Cohan, et al., J. Immunol. 156 (1996) 1714-21].

The physiological roles of DPP IV have not been completely elucidated. It has been thought that DPP IV plays a role, amongst others, in the regulation of fat intake, natriuresis, nociception, T-cell activation, regulation of blood glucose, and regulation of the digestive tract. DPP IV has been implicated in disease states such as HIV infection, diabetes, arthritis and certain cancers. For example, DPP IV activity and/or expression was found to be elevated in prostate [Wilson, et al., J Androl. 21 (2000) 220-6], colon [Fric, et al., Eur. J. Cancer Prev. 9 (2000):265-8], skin [Van den Oord, Br. J. Dermatol. 138 (1998) 615-21] and lung cancer [Sedo, et al., J Cancer Res. Clin. Oncol. 117 (1991) 249-53], and elevated DPP IV also has been found in patients having benign prostate hyperplasia. A high activity of DPP IV is also associated with membrane vesicles found in human, bovine, and equine ejaculate, where it is thought to play a role in the regulation of sperm motility and viability [Minelli A, et al., J Reprod. Fertil. 114 (1998) 237-43; Agrawal, et al., J Reprod. Fertil. 79 (1987) 409-19; Arienti, et al., FEBS Lett. 410 (1997) 343-6].

DPP IV also is being investigated for its role in type II diabetes because the glucagon-like peptide (GLP-1) can be a substrate for DPP IV cleavage, and some DPP IV inhibitors have demonstrated efficacy in animal models of diabetes. Additionally, DPP IV has been implicated in HIV infection due to its association with CD 26.

High levels of DPP IV expression have been reported for skin fibroblasts from human patients suffering from psoriasis, rheumatoid arthritis, and lichen planus [Raynaud, et al., J Cell Physiol. 151 (1992) 378]. Inhibition of DPP IV has been shown to increase release of TGF-β, a protein having neuroprotective properties. DPP IV inhibition itself has been implicated in cellular mechanisms relating to neurodegeneration [see PCT publication WO 01/34594].

It follows from the above that inhibitors of DPP IV may be useful as pharmaceuticals in the treatment of a range of medical conditions. In particular, they may be useful as immunosuppressants, anti-inflammatory agents, drugs that suppress tumor invasion and metastasis formation, drugs that inhibit HIV infectivity, regulators of blood glucose levels in patients suffering from diabetes, agents that affect sperm motility and viability useful both for contraception and in the reproduction of livestock, drugs for the treatment of dermatological disorders such as psoriasis, and as pharmaceuticals for the treatment of neurological disorder.

DPP IV inhibition has been studied in the treatment of autoimmune diseases such as diabetes, arthritis and multiple sclerosis. See PCT publications WO 97/40832 and WO 98/19998. Additionally, PCT publication WO 94/03055 discusses increasing production of hematopoietic cells with DPP IV inhibitors. PCT publication WO 95/11689 discloses the use of DPP IV inhibitors to block the entry of HIV into cells. U.S. Pat. No. 5,543,396 discloses the use of inhibitors (certain proline phosphonate derivatives) to treat tumor invasion. PCT publication WO 95/34538 mentions the use of certain serine protease inhibitors (such as certain DPP IV and PEP inhibitors) to treat inflammation-related neurological/autoimmune diseases like multiple sclerosis. Efficacy in experimental models of inflammatory disorders has also been described for compounds with DPP IV inhibitory activity, suggesting that such compounds may be useful in thr treatment of medical conditions such as rheumatoid arthritis and inflammatory bowel disorder. Augustyns et al. (Curr. Med. Chem.6 (1999) 311-327) and Hildebrandt et al. (Clinical Science 99 (2000) 93-104) review the wide therapeutic potential of various classes of DPP IV inhibitors.

DPP IV inhibitors based upon molecules that bear a resemblance to proline have been investigated in the field. For example, PCT publication WO 95/11689 discloses α-amino boronic acid analogs of proline. PCT publication WO 98/19998 discloses N-substituted 2-cyanopyrrolidines as DPP IV inhibitors. PCT publication WO 95/34538 discloses various proline containing compounds and phosphonate derivatives thereof. Proline phosphonate derivatives as inhibitors of DPP IV are also disclosed in U.S. Pat. No. 5,543,396. U.S. Pat. No. 6,172,081 discloses a series of tetrahydroisoquinoline 3-carboxaminde derivatives with potent DPP—W inhibitory activity; U.S. Pat. Nos. 6,166,063 and 6,107,317 disclose N-substituted 2-cyanopyrrolidines and 4-cyanothiazolidines, respectively. WO 95/15309 discloses various aminoacyl compounds as inhibitors of DPP IV. WO 01/68603 discloses a class of cyclopropyl-fused pyrrolidine derivatives as inhibitors of DPP IV. N-substituted 2-cyanopyrrole derivatives as inhibitors of DPP IV, and pharmaceutical compositions thereof, are taught for the treatment of various metabolic disorders in U.S. Patent Application Publication 2001/0031780.

In view of the needs of the art to provide new therapeutic products, methodologies, and uses, it is an object of the invention to provide novel inhibitors of dipetidyl peptidase. In accomplishing this object and other objects, there are provided, in accordance with one aspect of the invention, inhibitors of dipeptidyl peptidase IV which comprise modified N-substituted cyanopyrrolidine compounds of the following L general Formula I: 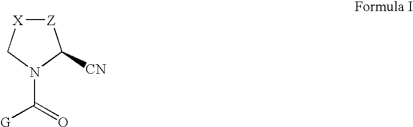
wherein the pyrrolidine ring formed by X, Z, N, and the carbon atoms to which they are attached, is saturated, or optionally contains one double bond;
X is selected from the group consisting of CH₂, CH, S, O, NH, N, C═O, CF₂, CF, CH—Y, and C—Y;
Z is selected from the group consisting of CH₂, CH, CF₂, CF, C—Y and CH—Y;
   wherein Y is halogen, hydroxy, or C₁-C₃ alkyloxy; and
   wherein one of X or Z must be CH₂; or CH if said pyrrolidine ring contains one double bond;
and where G is 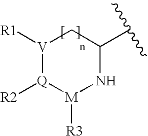
wherein M, Q, and V represent carbon atoms;
n is 0 or 1; and where either
   R1 and R2, taken together with V and Q, or
   R2 and R3, taken together with Q and M, form a 3-6 membered, saturated carbocyclic or heterocyclic ring which may contain one or two heteroatoms selected from the group consisting of O, S, and N.

In another aspect of this invention, there are provided inhibitors of DPP IV of the following general Formula II: 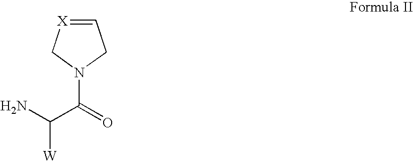
where X is as defined for Formula I above, and X may further be:
—S—CH₂—, —S—CH═, —CH₂—S—, (CH₂)₂, and —CH₂ —CH═,
and where W is either W′ or W″;
wherein W′ is a saturated cyclic hydrocarbon; and
W″ is a non-cyclic straight or branched chain alkyl group,
and the dashed bond symbol represents an optional bond.

In another aspect of this invention, there are provided inhibitors of DPP IV of the following general Formula IIa: 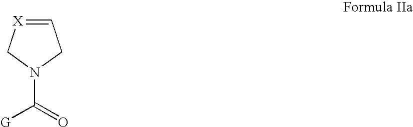
where the dashed bond symbol represents an optional bond,
X is defined as for Formula II above;
the substituent G is defined as for Formula I above;
   n in said substituent G is 0; and
   the 3-6 membered saturated ring in said substituent G is a carbocyclic ring.

In another aspect of this invention, there are provided inhibitors of DPP IV of the following general Formula III: 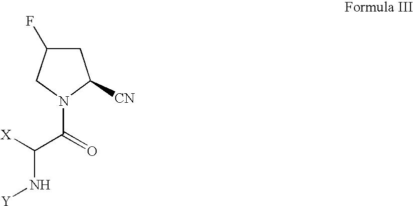
where X and Y may independently be H, or W as defined for Formula II above; provided that:
when Y is H, then X is W; and
when X is H, then Y is W; and
X and Y may not both be W.

In another aspect of this invention, there are provided inhibitors of DPP IV of the following general Formulae IVa and IVb: 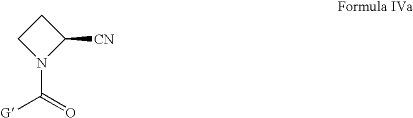
where G′ is a group G as defined for Formula 1 above; and where G′ may further be: 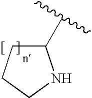
wherein n′ is 1 or 2. 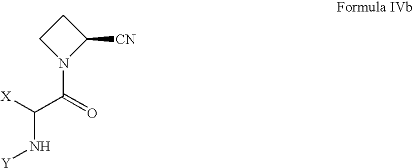
where X and Y may independently be H, or W as defined for Formula II above; provided that:
when Y is H, then X is W; and
when X is H, then Y is W; and
X and Y may not both be W.

In another aspect of this invention, there are provided compounds of the following general Formula V: 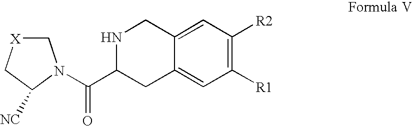
wherein X is CH₂, S, O, and C(CH₃)₂;
and R1 and R2 are independently selected from the group consisting of hydrogen, hydroxy, C₁-C₈ straight or branched chain alkyl, alkyl, alkoxy, aralkoxy, and halogen.

In yet another aspect of the invention, there are provided methods of treating a neurological disorder, comprising the step of administering to a patient in need of such treatment a therapeutically effective amount of a compound Formula V.

In another aspect of this invention, there are provided methods of treating a neurological disorder, comprising the step of administering to a patient in need of such treatment a therapeutically effective amount of a compound of the following general Formula VI: 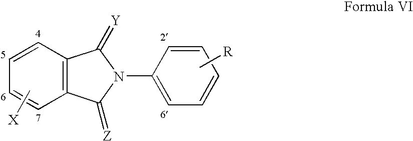
wherein the dashed bond symbol represents an optional bond;
X, if present, is a single substituent at one, or multiple substituents at several of positions 4-7; and is independently selected from the group consisting of nitro, amino, hydroxy, and halo;
Y and Z are independently O or S;
R is a single substituent at position 2′ or 6′, or two substituents at positions 2′ and 6′, and is independently selected from the group consisting of C₁-C₄ straight or branched chain alkyl, C₁-C₄ straight or branched alkoxy, C₁-C₄ straight or branched alkylthio, aminomethyl, and aminoethyl.

In another aspect of this invention, there are provided methods of treating a neurological disorder, comprising the step of administering to a patient in need of such treatment a therapeutically effective amount of a compound of the following general Formula VII: 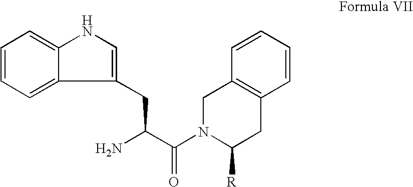
wherein R is a carboxy group, or an amino acid selected from the group consisting of Ala, Arg, Asp, Asn, Glu, Gln, Gly, His, Ile, Leu, Lys, Met, Phe, Pro, Ser, Thr, Trp, Tyr, Val, and Cys.

In another aspect of this invention, there are provided methods of treating a neurological disorder, comprising the step of administering to a patient in need of such treatment a therapeutically effective amount of a compound of the following general Formula VII: 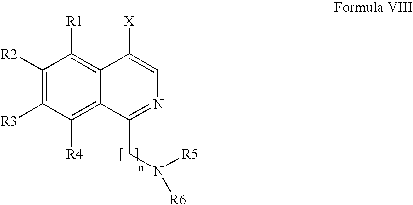
wherein n is 1 or 2;
R1, R2, R3, and R4 are independently hydrogen, methoxy, ethoxy, or propoxy;
R5 and R6 are independently hydrogen or methyl; and
X is —CO)—OEt; —CH═CH—(CO)—OEt; —CH₂—CH₂—(CO)—OEt; —COOH; —CONH₂; —CONH-Prop; —NH—(CO)—OEt; —CH₂—OH; CHO; or —CH₂—(CO)—OEt.

The compounds of Formula VIII are optionally in the form of di-HCl or di-TFA salts.

Another aspect of the present invention provides methods of treating a neurological disorder, comprising the step of administering to a patient in need of such treatment a therapeutically effective amount of a 2-cyanopyrrolidine compound of the following general Formula IX: 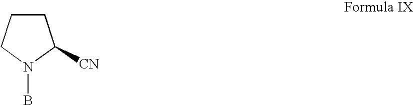
wherein at least one of the bonds in the 2-cyanopyrrolidine ring is a double bond; and
B is any alpha or beta amino acid connected to the ring with an amide or peptide bond.

Another aspect of the present invention provides methods of treating a neurological disorder, comprising the step of administering to a patient in need of such treatment a therapeutically effective amount of a compound of Formula IX, above, wherein B in said compound of Formula IX is B′ or B″: 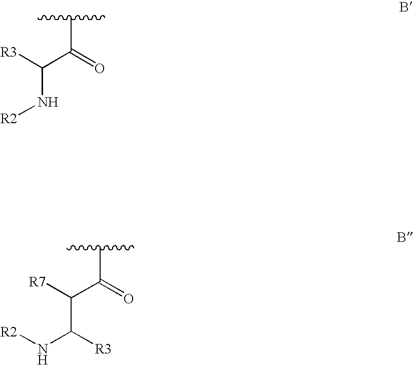
wherein R2 and R3 and R7 are independently C₁-C₁₀ alkyl, C₂-C₁₀ alkenyl, C₂-C₁₀ alkynyl, C₃-C₁₀ cycloalkyl, C₅-C₁₀ cycloalkenyl, aryl, heteroaryl, or hydrogen; provided, however, that R2 and R3 in B′ may not both be hydrogen; and that R2, R3, and R7 in B″ may not all be hydrogen; and where R7 in B″ may further be halogen, C₁-C₁₀ alkoxy, C₁-C₁₀ alkylthio, C₁-C₁₀ alkylamino, C₁-C₁₀ dialkylamino, hydroxymethyl, nitro, trifluoromethyl, trifluoromethoxy, trifluoromethylthio, N-hydroxyimino, cyano, carboxy, acetamido, hydroxy, sulfamoyl, or carbamoyl;
wherein said alkyl, alkenyl, alkynyl, cycloalkyl, or cycloalkenyl, are optionally and independently substituted with one or more R4; and wherein said aryl or heteroaryl are optionally and independently substituted with one or more R5; and wherein said aryl or heteroaryl in R3 is optionally fused to a C₃-C₁₀ cycloalkane;
R2 is optionally connected to R3, or R7 if present, by a single bond, or by a saturated or unsaturated bridge containing 1-3 atoms selected from the group consisting of carbon, nitrogen, oxygen, and sulphur; thus forming a ring, which is optionally fused to an aryl or heteroaryl, said aryl or heteroaryl being optionally substituted with one or several R5 independently,
R4, if present, is cycloalkyl, aryl optionally substituted with one or more R5 independently, heteroaryl optionally substituted with one or more R5 independently, amino optionally substituted with one or more R6 independently, —SO—R6, —SO₂—R6, —CO—R6, —COO—R6, —CONH—R6, —CON(R6), —O—R6, —S—R6, carboxy, acetamido, cyano, nitro, halogen, hydroxy, trifluoromethyl, trifluoromethoxy, sulfamoyl, carbamoyl, or hydroxymethyl;
R5, if present, is halogen, C₁-C₁₀ alkyl, C₁-C₁₀ alkoxy, C₁-C₁₀ alkylamino, C₁-C₁₀ dialkylamino, benzyl, benzyloxy, hydroxymethyl, nitro, trifluoromethyl, trifluoromethoxy, trifluoromethylthio, N-hydroxyimino, cyano, carboxy, acetamido, hydroxy, sulfamoyl, or carbamoyl;
R6, if present, is C₁-C₁₀ alkyl, C₂-C₁₀ alkenyl, C₂-C₁₀ alkynyl, C₃-C₁₀ cycloalkyl, or C₅-C₁₀ cycloalkenyl; wherein any one of said alkyl, alkenyl, alkynyl, cycloalkyl, or cycloalkenyl is optionally substituted with aryl, heteroaryl, benzyl, or phenethyl; said aryl or heteroaryl being optionally substituted with one or more R5 independently.

Another aspect of the present invention provides methods of treating a neurological disorder, comprising the step of administering to a patient in need of such treatment a therapeutically effective amount of a compound of Formula Xa: 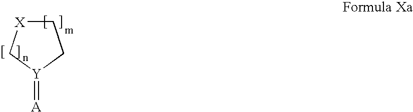
wherein X is CH₂, S, O, SO, SO₂, NH, or N(C₁-C₆ alkyl);
Y is N, CH, or C;
n is 1 or 2;
m is 0, 1, or 2;
the dashed bond symbol represents an optional bond;
and A is either:
   an alpha-amino acyl group derived from an alpha-amino acid bearing a mono- or bicycloaliphatic side chain, said side chain being saturated or partially saturated, and optionally containing one or more heteroatoms;
or A is:
   a beta-amino acyl group of the formula 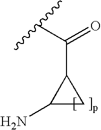
   wherein p is 1-6, and the ring in said beta-amino acyl group is saturated or partially saturated, and optionally contains one or more heteroatoms;
wherein the 1′ carbonyl group in said alpha- or beta-amino acyl groups is optionally replaced by CH or CF.

Another aspect of the present invention provides methods of treating a neurological disorder, comprising the step of administering to a patient in need of such treatment a therapeutically effective amount of a compound of Formula Xb: 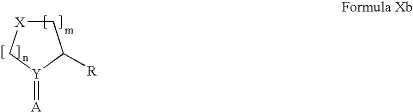
wherein X, Y, m, and n are as defined for Formula Xa above;
R is CN, C═C—R7, or CH═N—R8;
R7 is hydrogen, fluoro, nitro, C₁-C₆ alkyl, C₁-C₆ alkoxy, C₁-C₆ alkoxycarbonyl, or C₁-C₆ alkanoyl;
R8 is phenyl, hydroxy, C₁-C₆ alkoxy, —O—(CO)—(C₁-C₆ alkyl), or benzyloxy;
A is as defined for Formula Xa above, and in addition may be derived from any L-alpha-amino acid bearing a lipophilic side chain.

Another aspect of the present invention provides methods of treating a neurological disorder, comprising the step of administering to a patient in need of such treatment a therapeutically effective amount of a compound of Formula Xc: 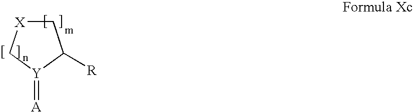
wherein X, Y, m, and n are as defined for Formula Xa above;
R is CHO or B(OH)₂;
A is a beta amino acyl group as defined for Formula Xa above.

Another aspect of the present invention provides methods of treating a neurological disorder, comprising the step of administering to a patient in need of such treatment a therapeutically effective amount of a compound of Formula Xd: 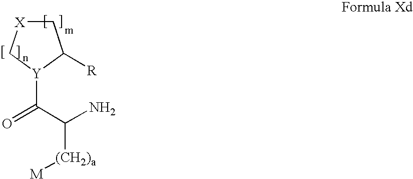
wherein X, Y, m, and n are as defined for Formula Xa above;
R is H, CN, C≡C—R7, or CH═N—R8, wherein R7 and R8 are as defined for Formula Xb, above;
a is 1-5;
M is:
   —COO——(CH₂)_{b}—(R4)_{q}—R3,
   —CONH—(CH₂)_{b}—R4)_{q}—R3,
   —CONCH₃—(CH₂)_{b}—R4)_{q}—R3,
   —SO₂—NH—(CH₂)_{b}—(R4)_{q}—R3, or
   —SO₂—NCH₃—(CH₂)_{q}—R4)_{q}—R3;
   wherein b is 0-12; q is 0-5;
   R4 is Z—NH—(CH₂)_{c}- or NH—Z—(CH₂)_{c}—;
   wherein c is 1-12; and
   Z is CO, CH₂, or SO₂; and
   R3 is
   COOH,
   —(COO)—(C₁-C₈ alkyl or fluoroalkyl),
   —COO)—(C₁-C₈ cycloalkyl),
   —(COO)-aryl,
   —(COO)-heteroaryl,
   CONH₂,
   CONHNH₂,
   CONR5R6,
   CONNR5R6,
   PO₃H,
   PO₃—C₁-C₈ alkyl or fluoroalkyl),
   PO₃—C₁-C₈ cycloalkyl), PO₃-aryl,
   PO₃-heteroaryl,
   SO₃H,
   SO₂NH₂,
   SO₂NR5R6,
   OH,
   OR5,
   NH₂,
   NR5R6,
   NHCOOR5,
   NHSO₂NR5R6,
   NHCOR5,
   NHSO₂R5,
   NH—CH(:NR5)NR5R6,
   NHCONR5R6,
   aryl, or heteroaryl, wherein said aryl or heteroaryl is mono- or bicyclic, the individual rings consisting of 5-6 members, and being optionally substituted with one or more substituents selected from the group consisting of F, Cl, I, Br, OH, OR5, NO₂, SO₃H, SO₂NH₃, SO₂NR5R6, NH₂, NR5R6, COOR5, CF₃, CN, CONH₂, CONR5R6, NHCOOR5, CH(:NR5)NR5R6, NH—CH(:NR5)NR5R6 and R5;
   sugar, which is attached via an ether or a glycosidic bond;
   CO-aminosugar which is attached via its amino group;
   NHCO-aminosugar, or
   NHCS-aminosugar, wherein the term “sugar” in said sugar, CO-aminosugar, NHCO-aminosugar, or NHCS-aminosugar groups refers to any carbohydrate or oligosaccharide;
   wherein R5 and R6 are independently selected from H, C₁-C₈ straight or branched chain alkyl, C₁-C₈ straight or branched chain fluoroalkyl, C₃-C₈ cycloalkyl, and aryl, heteroaryl, or alkylheteroaryl of up to 11 atoms;
   or wherein R5 and R6 together optionally form a 3-8-membered carbocyclic chain.

Another aspect of the present invention provides methods of treating a neurological disorder, comprising the step of administering to a patient in need of such treatment a therapeutically effective amount of a compound of Formula Xe: 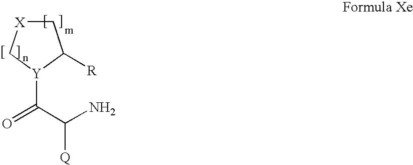
wherein X, Y, m, and n are as defined for Formula Xa above;
R is as defined for Formula Xd above;
Q is a group selected from 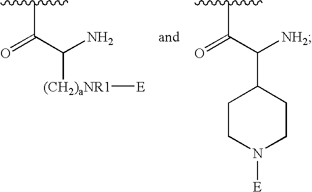
R1 is H or CH₃;
E is
   —(CO)—(CH₂)_{b}(R4)_{q}—R3,
   —CH₂—(CH₂)_{b}—R4)_{q}—R3; or
   —SO₂—(CH₂)_{b}—(R4)_{q}—;
wherein a, b, q, R3, and R4 are as defined for Formula Xd, above.

Another aspect of the present invention provides methods of treating a neurological disorder, comprising the step of administering to a patient in need of such treatment a therapeutically effective amount of a compound of Formula Xf: 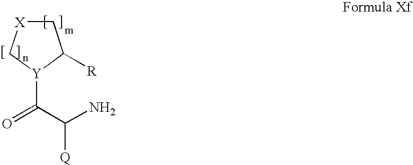
wherein X, Y, m, and n are as defined for Formula Xa above;
R is as defined for Formula Xd above;
Q is a group selected from 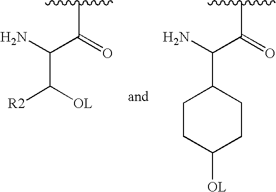
L is
   —(CH₂)_{d}—(CO)ᵣ—(CH₂)_{b}—(R4)_{q}—R3, or
   —(CH₂)ₑ—NR1 —(CH₂)_{b}—(R4)_{q}—R3;
R1 and R2 are independently H or CH₃;
r is 0 or 1;
d is 0-4;
e is 2-4; and
b, q, R3 and R4 are as defined for Formula Xd, above.

Another aspect of the present invention provides methods of treating a neurological disorder, comprising the step of administering to a patient in need of such treatment a therapeutically effective amount of a compound of Formula XI: 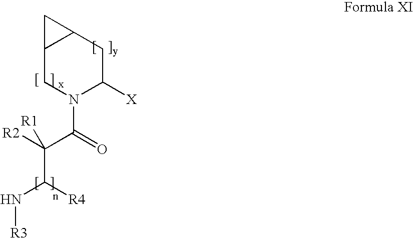
wherein x and y are independently 0 or 1, provided that only one of x and y can be 0;
n is 0 or 1;
X is H or CN;
R1, R2, R3, and R4 are independently selected from hydrogen, alkyl, alkenyl, alkynyl, cycloalkyl, cycloalkylalkyl, bicycloalkyl, tricycloalkyl, alkylcycloalkyl, hydroxyalkyl, hydroxyalkylcycloalkyl, hydroxycycloalkyl, hydroxybicycloalkyl, hydroxytricycloalkyl, bicycloalkylalkyl, alkylthioalkyl, arylalkylthioalkyl, cycloalkenyl, aryl, aralkyl, heteroaryl, heteroarylalkyl, cycloheteroalkyl or cycloheteroalkylalkyl; all optionally substituted through available carbon atoms with 1, 2, 3, 4 or 5 groups selected from hydrogen, halo, alkyl, polyhaloalkyl, alkoxy, haloalkoxy, polyhaloalkoxy, alkoxycarbonyl, alkenyl, alkynyl, cycloalkyl, cycloalkylalkyl, polycycloalkyl, heteroarylamino, arylamino, cycloheteroalkyl, cycloheteroalkylalkyl, hydroxy, hydroxyalkyl, nitro, cyano, amino, substituted amino, alkylamino, dialkylamino, thiol, alkylthio, alkylcarbonyl, acyl, alkoxycarbonyl, aminocarbonyl, alkynylaminocarbonyl, alkylaminocarbonyl, alkenylaminocarbonyl, alkylcarbonyloxy, alkylcarbonylamino, arylcarbonylamino, alkylsulfonylamino, alkylaminocarbonylamino, alkoxycarbonylamino, alkylsulfonyl, aminosulfinyl, aminosulfonyl, alkylsulfinyl, sulfonamido or sulfonyl;
and wherein R1 and R3 may optionally be taken together to form a group —CR5R6)ₘ— where m is 2 to 6, and R5 and R6 are the same or different and are independently selected from hydroxy, alkoxy, H, alkyl, alkenyl, alkynyl, cycloalkyl, halo, amino, substituted amino, cycloalkylalkyl, cycloalkenyl, aryl, arylalkyl, heteroaryl, heteroarylalkyl, cycloheteroalkyl, cycloheteroalkylalkyl, alkylcarbonylamino, arylcarbonylamino, alkoxycarbonylamino, aryloxycarbonylamino, alkoxycarbonyl, aryloxycarbonyl, or alkylaminocarbonylamino,
or R1 and R4 may optionally be taken together to form —CR7R8)ₚ—wherein p is 2 to 6, and R7 and R8 are the same or different and are independently selected from hydroxy, alkoxy, cyano, H, alkyl, alkenyl, alkynyl, cycloalkyl, cycloalkylalkyl, cycloalkenyl, halo, amino, substituted amino, aryl, arylalkyl, heteroaryl, heteroarylalkyl, cycloheteroalkyl, cycloheteroalkylalkyl, alkylcarbonylamino, arylcarbonylamino, alkoxycarbonylamino, aryloxycarbonylamino, alkoxycarbonyl, aryloxycarbonyl, or alkylaminocarbonylamino,
or optionally R1 and R3 together with 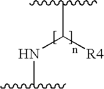
   form a 5 to 7 membered ring containing a total of 2 to 4 heteroatoms selected from N, O, S, SO, or SO₂;
or optionally R1 and R3 together with 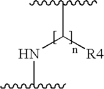
   form a 4 to 8 membered cycloheteroalkyl ring wherein the cycloheteroalkyl ring has an optional aryl ring fused thereto or an optional 3 to 7 membered cycloalkyl ring fused thereto.

The compounds for use in the methods of this aspect of the invention are optionally in the form of a salt with a pharmaceutically acceptable acid or base.

In yet another aspect of this invention, there is provided a method of treating medical conditions which can be alleviated by inhibition of DPP IV, comprising administering to a mammal in need of such treatment a therapeutically effective amount of a compound of Formulae I-IV, or of a pharmaceutically acceptable derivative thereof.

The present invention further provides a method of inhibiting DPP IV in a mammal, comprising administering to a mammal in need thereof a therapeutically effective amount of a compound of Formulae I-IV, or of a pharmaceutically acceptable derivative thereof.

Also included in the present invention are pharmaceutical compositions useful in inhibiting DPP IV, which comprise a therapeutically effective amount of one or several compounds of Formulae I-IV, or of a pharmaceutically acceptable derivative thereof, and a pharmaceutically acceptable carrier, diluent, or excipient.

Compounds of Formulae I-XI may be prepared or formulated as a salt or derivative for some uses, including pharmaceutical and tissue or cell culture uses. As used herein, the compounds of this invention are defined to include pharmaceutically acceptable derivatives. A “pharmaceutically acceptable derivative” denotes any pharmaceutically acceptable salt, ester, thioester, amide, or salt of such ester, thioester, or amide, of a compound of this invention or any other compound which, upon administration to an animal or human patient, is capable of providing (directly or indirectly) a compound of this invention, or a metabolite or residue thereof, characterized by the ability to inhibit DPP IV and/or its usefulness in treating or preventing a medical disorder. Examples of medical disorders within the scope of this aspect of the invention are given below. As stated above, the compounds of the invention can also be part of a composition comprising one or more compounds of Formulae I-XI.

The term “alkyl” refers to optionally substituted straight or branched chain hydrocarbon groups having 1 to 8 carbon atoms, preferably 1 to 5 carbons. Exemplary unsubstituted alkyl groups include methyl, ethyl, propyl, butyl, pentyl, hexyl, heptyl, octyl, the various branched chain isomers thereof, such as isopropyl, t-butyl, isobutyl, isohexyl, 4,4-dimethylpentyl, 2,2,4-trimethylpentyl and the like. Substituted alkyl groups include said alkyl groups substituted by one or more substituents selected from halogen, alkoxy, cycloalkyl, hydroxy, carboxy, —CONR₃R₄, —NR₃R₄ (where R₃ and R₄ are independently hydrogen or alkyl), nitro, cyano or thiol.

The term “alkoxy” refers to any of the above alkyl groups linked to an oxygen atom.

The term “cycloalkyl” refers to saturated cyclic hydrocarbon groups containing 3 to 7 ring carbons with cyclopropyl, cyclopentyl and cyclohexyl being preferred.

The term “halogen” or “halo” refers to chlorine, bromine and fluorine.

The term “aryl” refers to monocyclic or bicyclic aromatic hydrocarbon groups having 6 to 12 carbon atoms in the ring portion, such as phenyl, naphthyl, tetrahydronaphthyl or biphenyl groups, each of which may optionally be substituted by one to four substituents such as alkyl, halo, hydroxy, alkoxy, amino, thiol, nitro, cyano, carboxy and the like.

The term “aralkoxy” refers to an aryl group bonded to an alkoxy group.

Specifically, as used herein, the term “saturated cyclic hydrocarbon” means saturated cyclic hydrocarbon groups containing 3 to 7 ring carbons, and further includes fused, bridged, or spirocyclic bicyclic saturated hydrocarbon groups containing 6-14 ring carbons.

“Non-cyclic straight or branched chain alkyl group” means a C₂-C₉, preferably C₃-C₆, hydrocarbon chain, for example t-butyl, 4,4-dimethylpentyl, 2,2,4-trimethylpentyl, octyl, and the like.

Insofar as its preparation is not specifically mentioned or incorporated by reference herein, a compound used as a starting material for the synthesis of the compounds of this invention is known or may be prepared from known compounds, or in a known manner, or analogously to known methods, or analogously to the methods described herein, as will be appreciated by one skilled in the art. The compounds of the invention can be produced as a mixture of isomers or racemic mixtures or as optically pure compounds. Methods for separating stereoisomers known in the art can also be used to enrich mixtures for one or more compounds. The compositions of the invention may similarly contain mixtures of stereoisomers, mixtures of one or more stereoisomers, or be enriched for one or more stereoisomers. All of these forms are specifically included in this invention and are intended to be included in the claims.

The compounds of Formulae I-XI possess important utility as pharmaceuticals, especially in the treatment of medical conditions which can be alleviated by inhibition of DPP IV. Examples of such medical conditions are given below. However, the methods of the present invention are not limited to the treatment of such medical conditions alone. Thus, the ability of the compounds of the instant invention to bind to, and inhibit DPP IV further renders the compounds of Formulae I-XI useful in a variety of diagnostic and research applications. For example, in vitro techniques can be used to identify and characterize cellular components or chemical compounds that interact with DPP IV in a cell-free environment, as would be the case when a compound of Formulae I-XI is used to competitively bind to, or inhibit, DPP IV in the presence of such other chemical compound or cellular component. Further, compounds of Formulae I-XI may be labeled with a suitable radioisotope and in such form utilized for determining the cellular or tissue distribution of DPP IV in a given tissue sample, or utilized as a diagnostic medical imaging agent for the visualization of e.g. tumors which express high levels of DPP IV.

Another aspect of this invention provides methods for treating a medical condition in a patient in need of such treatment. Medical conditions to be treated with the compounds and compositions of this invention according to these methods include neurological disorders, diabetes, hyperglycemia, obesity, atherosclerosis, polycystic ovary syndrome, arthritis, autoimmune disorders, AIDS, osteoporosis, chronic inflammatory bowel disease, AIDS, metastatic cancer, and cutaneous disorders such as psoriasis and lichen planus. The instant compounds are further useful as immunosuppressants in allograft recipients, contraceptive agents affecting sperm function, and for the treatment of anorexia.

Neurological disorders to be treated according to the methods of this invention, when present in an animal, including humans, can be neurodegenerative disorders, neuropathic disorders, neurovascular disorders, traumatic injury of the brain, spinal cord, or peripheral nervous system, demyelinating disease of the central or peripheral nervous system, metabolic or hereditary metabolic disorder of the central or peripheral nervous system, or toxin-induced- or nutritionally related disorder of the central or peripheral nervous system. When present in a human, a neurodegenerative disorder can be, for example, Parkinson's disease, Alzheimer's disease, amyotrophic lateral sclerosis (ALS), Huntington's disease, cerebellar ataxia, or multisystem atrophy including, for example, olivopontocerebellar degeneration, striatonigral degeneration, progressive supranuclear palsy, Shy-Drager syndrome, spinocerebellar degeneration and corticobasal degeneration. A demyelinating disease can be, for example, multiple sclerosis, Guillain-Barré syndrome, or chronic inflammatory demyelinating polyradiculoneuropathy. A neurovascular disorder can be global cerebral ischemia, spinal cord ischemia, ischemic stroke, cardiogenic cerebral embolism, hemorrhagic stroke, lacunar infarction, multiple infarct syndromes including multiple infarct dementia, or any disorder resulting in ischemia or ischemia/reperfusion injury of the central nervous system. Traumatic injury of the central or peripheral nervous system can be, for example, concussion, contusion, diffuse axonal injury, edema, and hematoma associated with craniocerebral or spinal trauma, or axonal or nerve sheath damage associated with laceration, compression, stretch, or avulsion of peripheral nerves or plexi, and further includes damage to central nervous tissue or peripheral or visceral nervous tissue caused during surgery, such as damage to the major pelvic ganglion and/or cavernous nerve caused during prostate surgery. A neuropathic disorder can be, for example, diabetic neuropathy, uremic neuropathy, neuropathy related to therapy with drugs such as phenyloin, suramin, taxol, thalidomide, vincristine or vinblastine; or neuropathy/encephalopathy associated with infectious disease, such as, for example, encephalopathy related to HIV, rubella virus, Epstein-Barr virus, herpes simplex virus, toxoplasmosis, prion infection. A metabolic disorder of the central nervous system can be, for example, status epilepticus, hypoglycemic coma, or Wilson's disease.

A compound of this invention can be administered to an animal or human patient by itself or in pharmaceutical compositions where it is mixed with suitable carriers or excipients, at doses to treat or ameliorate various conditions. The compounds according to the present invention preferably have sufficient stability, potency, selectivity, solubility and availability to be safe and effective in treating diseases, injuries and other abnormal medical conditions or insults, including medical conditions of, and insults to, the central nervous system, the peripheral nerves, and other organs. A therapeutically effective dose refers to that amount of the compound sufficient to effect an activity in a nerve or neuronal cell, to produce a detectable change in a cell or organism, or to treat a disorder in a human or other mammal. The word “treat” in its various grammatical forms as used in relation to the present invention refers to preventing, curing, reversing, attenuating, alleviating, minimizing, suppressing, ameliorating or halting the deleterious effects of a disease state, disease progression, injury, wound, ischemia, disease causative agent (e.g., bacteria, protozoans, parasites, fungi, viruses, viroids and/or prions), surgical procedure or other abnormal or detrimental condition (all of which are collectively referred to as “disorders,” as will be appreciated by the person of skill in the art). A “therapeutically effective amount” of a compound according to the invention is an amount that can achieve effective treatment, and such amounts can be determined in accordance with the present teachings by one skilled in the art.

The methods of the present invention comprise (i.) administration of a compound of Formulae I-XI, where the compound is itself therapeutically active in the treatment of the targeted medical condition, or (ii.) administration of a prodrug of a compound of Formulae I-XI, wherein such prodrug is any compound which is capable of undergoing metabolic conversion to a compound of Formulae I-XI following administration, or (iii.) administration of a compound of Formulae I-XI where the compound is capable of undergoing metabolic conversion to a metabolite following administration, and where the metabolite is therapeutically active in the treatment of the targeted medical condition, or (iv.) administration of a metabolite of a compound of Formulae I-XI, where the metabolite is therapeutically active in the treatment of the targeted medical condition. Thus, the use of a compound of Formulae I-XI in the methods of the present invention explicitly includes not only the use of the compound itself, but also the modifications ii, iii, and iv discussed in this paragraph, and all such modifications are explicitly intended to be within the scope of the following claims.

Therapeutically effective doses may be administered alone or as adjunctive therapy in combination with other treatments. Techniques for the formulation and administration of the compounds of the instant application may, for example, be found in Remington's Pharmaceutical Sciences, Mack Publishing Co., Easton, Pa., 18^{th} edition (1990), and subsequent editions thereof.

Suitable routes of administration may, for example, include oral, rectal, transmucosal, buccal, or intestinal administration; parenteral delivery, including intramuscular, subcutaneous, intramedullary injections, as well as intrathecal, direct intraventricular, intravenous, intraperitoneal, intranasal, or intraocular injections, and optionally in a depot or sustained release formulation. Furthermore, one may administer the agent of the present invention in a targeted drug delivery system, for example in a liposome coated with an antibody. The liposomes will be targeted to and taken up selectively by cells expressing the appropriate antigen.

The pharmaceutical compositions of the present invention may be manufactured in a manner that is itself known, e.g., by means of conventional mixing, dissolving, emulsifying, encapsulating, entrapping, or lyophilizing processes. Pharmaceutical compositions for use in accordance with the present invention thus may be formulated in conventional manner using one or more physiologically acceptable carriers comprising excipients and auxiliaries, which facilitate processing of the active compounds into preparations, which can thus be used pharmaceutically.

For injection, the compounds of the invention may be formulated in aqueous solutions, preferably in physiologically compatible buffers, such as Hank's solution, Ringer's solution, or physiological saline buffer. For transmucosal or buccal administration, penetrants appropriate to the barrier to be permeated may be used in the formulation. Such penetrants are known in the art.

For oral administration, the compounds can be formulated readily by combining the active compounds with pharmaceutically acceptable carriers, well known to those in the art. Such carriers enable the compounds of the invention to be formulated as tablets, pills, capsules, liquids, quick-dissolving preparations, gels, syrups, slurries, suspensions and the like, for oral ingestion by a patient to be treated. Pharmaceutical preparations for oral use of the compounds of this invention can be obtained by employing a solid excipient, optionally grinding a resulting mixture, and processing the mixture of granules, after adding suitable auxiliaries, if desired, to obtain tablets. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP).

In general, the pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols. If desired, disintegrating agents may be added, such as the cross-linked polyvinyl pyrrolidone, agar, or alginic acid or a salt thereof such as sodium alginate or a number of others disintegrants (see, for example, Remington's Pharmaceutical Sciences, Mack Publishing Co., Easton, Pa., 18^{th} edition (1990), and subsequent editions thereof).

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, pressurized air, or other suitable gas or mixture. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents, which increase the solubility of the compounds to allow for the preparation of highly concentrated solutions. Alternatively, the active ingredient may be in powder form for reconstitution with a suitable vehicle, e.g., sterile pyrogen-free water, before use.

The compounds may also be formulated in rectal compositions such as suppositories, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compounds of the invention may further be formulated in pharmaceutical or cosmetic compositions for topical application to the skin in the form of an aqueous, alcoholic, aqueous/alcoholic or oily solution, or of a dispersion of the lotion or serum type, of an emulsion having a liquid or semi-liquid consistency of the milk type, obtained by dispersion of a fatty phase in an aqueous phase (O/W) or vice versa (W/O), or of a suspension or of an emulsion with a soft consistency of the aqueous or anhydrous gel, foam or cream type, or, alternatively, of microcapsules or microparticles, or of a vesicular dispersion of ionic and/or nonionic type, or may further be administered in the form of an aerosol composition comprising a pressurized propellent agent. The compounds of the invention, for use in the treatment of a cutaneous disorder such as, for example, psoriasis or lichen planus, can also be formulated into various compositions for hair care and, in particular, shampoos, hair-setting lotions, treating lotions, styling creams or gels, dye compositions (in particular oxidation dyes), optionally in the form of color-enhancing shampoos, hair-restructuring lotions, permanent-wave compositions, and the like. Pharmaceutical or cosmetic compositions comprising compounds of the invention can also contain additives and adjuvants which are conventional in the cosmetics field, such as gelling agents, preservatives, antioxidants, solvents, fragrances, fillers, screening agents, odor absorbers and colorants. The amounts of these different additives and adjuvants are those typically employed in the cosmetics field and range, for example, from 0.01% to 20% of the total weight of the composition, preferably 0.1% to 10%, and more preferably 0.5% to 5%. In addition to one or several compounds of the invention, compositions for topical application may further contain additional agents already known in the art to promote hair growth or to prevent or retard hair loss, such as, without limitation, tocopherol nicotinate, benzyl nicotinate or 2,4-diamino-6-piperidinopyrimidine 3-oxide, or may contain other active agents such as antibacterial agents, antiparasitic agents, antifungal agents, antiviral agents, anti-inflammatory agents, antipruriginous agents, anaesthetic agents, keratolytic agents, antiseborrhoeic agents, antidandruff agents, or antiacne agents. The cosmetic or pharmaceutical compositions according to the invention can be topically applied onto the affected areas of the scalp and skin of an individual and optionally maintained in contact for a number of hours and optionally rinsed. It is possible, for example, to apply the composition containing an effective amount of at least one compound of the invention in the evening, to retain the composition in contact overnight and optionally to shampoo in the morning. These applications can be repeated daily for one or a number of months, depending on the particular individuals involved.

Liposomes and emulsions are well known examples of delivery vehicles or carriers for hydrophobic drugs. Certain organic solvents such as dimethylsulfoxide also may be employed. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for stabilization may be employed.

Pharmaceutical compositions suitable for use in the present invention include compositions wherein the active ingredients are contained in an effective amount to achieve their intended purpose, to effect a therapeutic benefit, or to effect a detectable change in the function of a cell, tissue, or organ. More specifically, a therapeutically effective amount means an amount effective to prevent the development of or to alleviate the existing symptoms of the subject being treated. Determining the effective amount is well within the capability of those skilled in the art, especially in light of the detailed disclosure provided herein.

The compounds of this invention may be administered in conjunction with, or formulated in pharmaceutical compositions together with, one or several additional therapeutic agents. Such additional therapeutic agents are themselves known in the art, and the specific agent employed together with the compounds of Formulae I-XI in this embodiment of the invention depend on the medical condition to be treated. Medical conditions wherein the compounds of Formulae I-XI are useful as therapeutic agents include diabetes, hyperglycemia, impaired glucose homeostasis, impaired glucose tolerance, infertility, polycystic ovary syndrome, growth disorders, frailty, arthritis, allograft rejection in transplantation, autoimmune diseases (such as scleroderma and multiple sclerosis), various immunomodulatory diseases (such as lupus erythematosis or psoriasis), AIDS, intestinal diseases (such as necrotizing enteritis, microvillus inclusion disease or celiac disease), chemotherapy-induced intestinal mucosal atrophy or injury, osteoporosis, Syndrome X dysmetabolic syndrome, diabetic complications, hyperinsulinemia, obesity, atherosclerosis and related diseases, as well as inflammatory bowel disease (such as Crohn's disease and ulcerative colitis), obesity, atherosclerosis, and neurodegenerative disorders. The instant compounds are further useful as immunosuppressants in allograft recipients, contraceptive agents affecting sperm function, and for the treatment of anorexia It follows that additional therapeutic agents to be used in combination with the compounds of this invention are selected from such agents known in the art to possess therapeutic utility in the medical condition to be treated, In the treatment of diabetes, for example, compounds of Formulae I-XI may be used in combination with one or more other types of antidiabetic agents which may be administered by any of the herein described routes in the same dosage form, or in a separate dosage form. Such other types of antidiabetic agents which may be used in combination with the compounds of this invention are themselves known in the art, and include, for example, biguanides, sulfonyl ureas such as glyburide, glucosidase inhibitors, thiazolidinediones such as troglitazone (Rezulin®), glycogen phosphorylase inhibitors, and insulin. In the treatment of inflammatory disorders, for example, compounds of Formulae I-XI may be used in combination with one or several agents which themselves have therapeutic utility in that condition, such as aspirin, indomethacin, ibuprofen, ketoprofen, naproxen sodium, celecoxib (Celebrex®), or rofexocib (Vioxx®).

Toxicity and therapeutic efficacy of the compounds or compositions can be determined by standard pharmaceutical, pharmacological, and toxicological procedures in cell cultures or experimental animals. For example, numerous methods for determining the LD₅₀ (the dose lethal to 50% of the population) and the ED₅₀ (the dose therapeutically effective in 50% of the population) exist. The dose ratio between toxic and therapeutic effects is the therapeutic index, which can be expressed as the ratio between LD₅₀ and ED₅₀. Compounds and compositions exhibiting high therapeutic indices are preferred. The data obtained from cell culture assays or animal studies can be used in formulating a range of dosages for use in humans, as has long been established in the art [see, e.g., Fingl et al., in The Pharmacological Basis of Therapeutics, Ch. 1 p. 1(1975)].

The compounds of the present invention may be administered by a single dose, multiple discrete doses or continuous infusion. Because the compounds preferably are non-peptidic, easily diffusible and relatively stable, they can be well-suited to continuous infusion.

Dose levels on the order of about 0.1 mg to about 10,000 mg of the active ingredient are useful in the treatment of the above conditions, with preferred levels being about 0.1 mg to about 1,000 mg, and 1 mg to about 1000 mg. The specific dose level, and thus the therapeutically-effective amount, for any particular patient will vary depending upon a variety of factors, including the activity of the specific compound employed and its bioavailability at the site of drug action; the age, body weight, general health, sex and diet of the patient; the time of administration; the rate of excretion; drug combination; the severity of the particular disease being treated; and the form of administration. Typically, in vitro dosage-effect results provide useful guidance on the proper doses for patient administration. Studies in animal models also are helpful. The considerations for determining the proper dose levels are available to the skilled person.

Suitable compounds of this invention can be administered in lyophilized form. In this case, 1 to 1000 mg, preferably 20-500 mg, of a compound of the present invention may be lyophilized in individual vials, together with a carrier and a buffer, such as mannitol and sodium phospshate. The compound may be reconstituted in the vials with bacteriostatic water before administration.

In treating a neurodegenerative disorder, for example, the compounds of the present invention are preferably administered orally, rectally, or parenterally 1 to 6 times daily, and may follow an initial bolus dose of higher concentration. In treating a cutaneous disorder, such as psoriasis or lichen planus, for example, the compounds of the present invention are preferably administered topically or orally one—4 times daily.

For the compounds, methods, and uses of the present invention, any administration regimen regulating the timing and sequence of drug delivery can be used and repeated as necessary to effect treatment. Such regimen may include pretreatment and/or co-administration with additional therapeutic agents.

The following description should not be taken as a limitation on the scope of the invention, and all embodiments and examples given are merely illustrative of the invention. Additional aspects of the invention can be devised by reference to this disclosure as a whole in combination with the references cited and listed throughout and at the end of the specification and the knowledge of one skilled in the art. All of the references cited and listed can be relied on, in their entirety, to allow one to make and use these additional aspects of the invention:

### EXEMPLARY COMPOUNDS

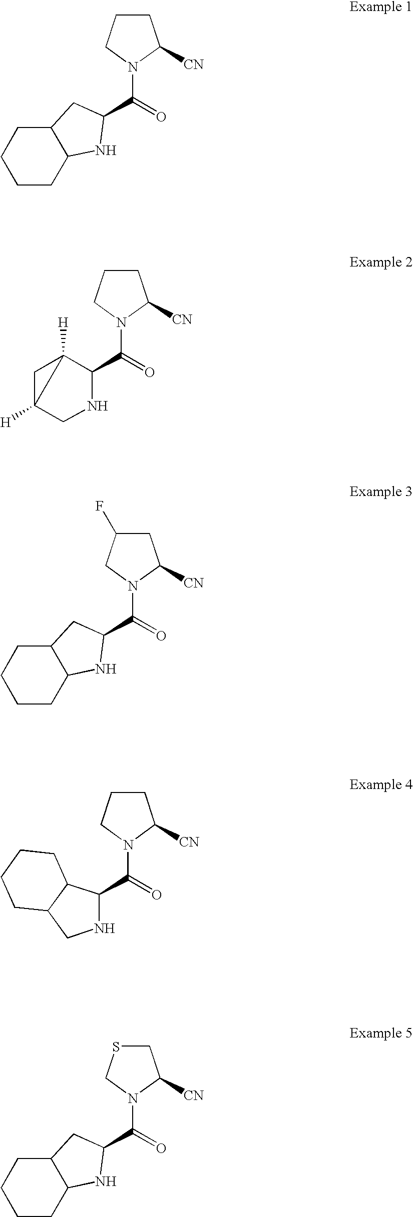

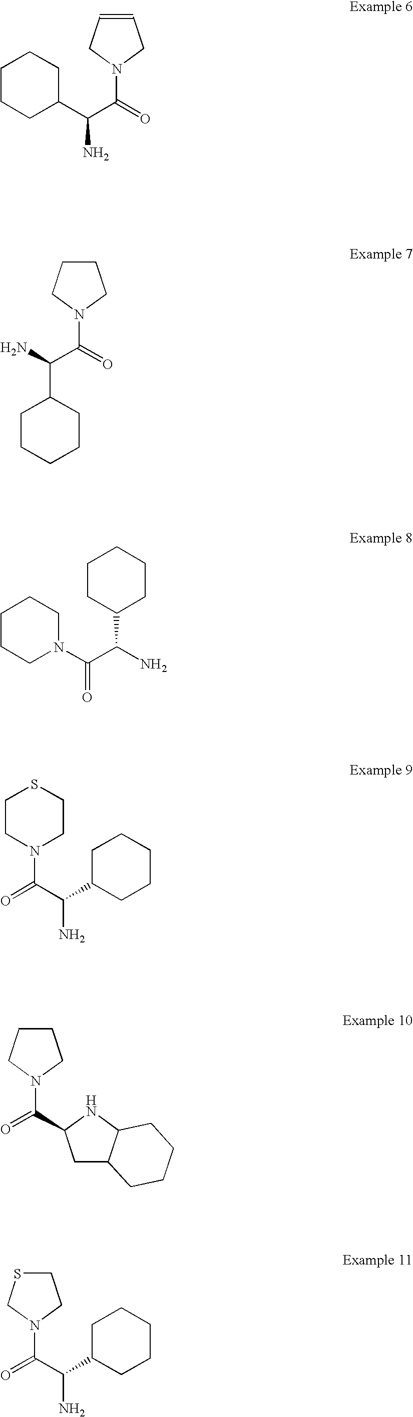

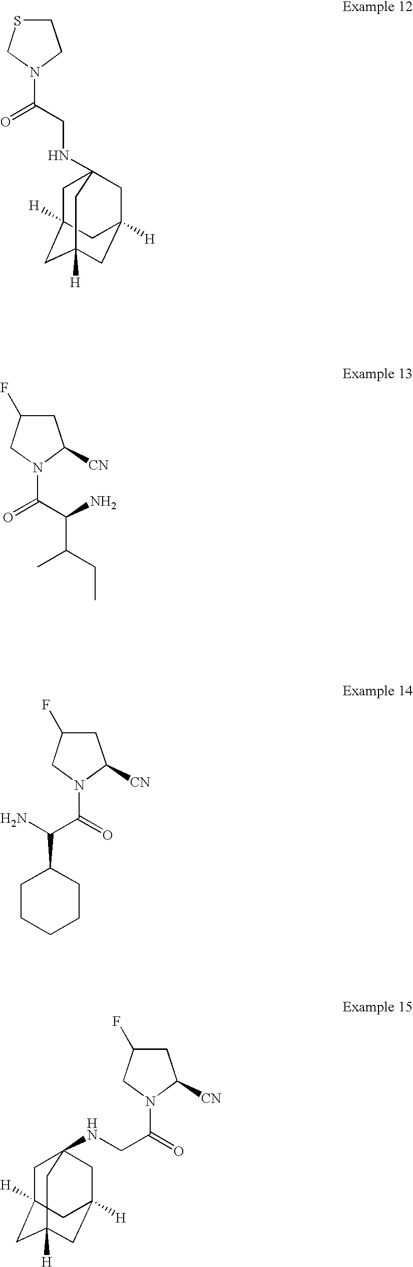

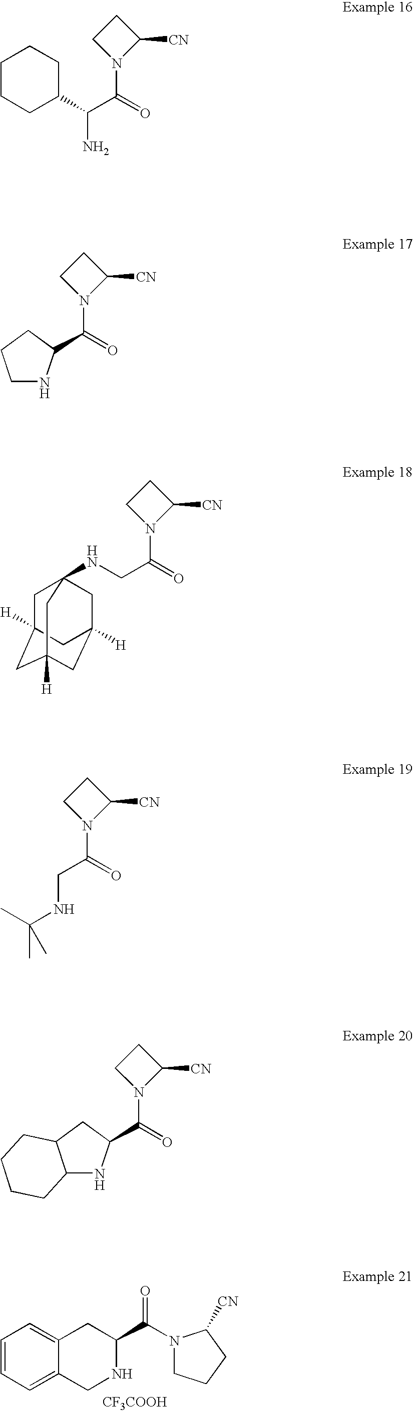

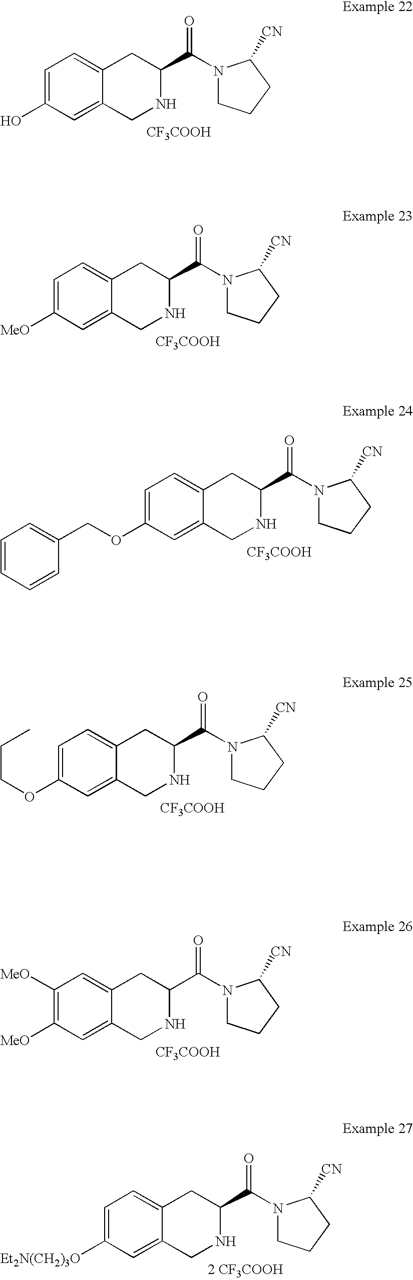

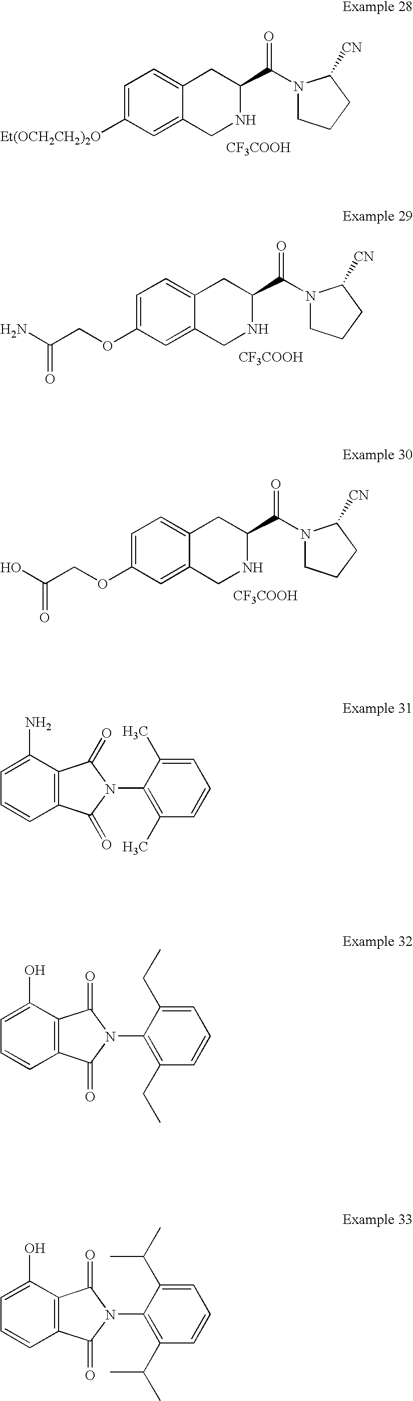

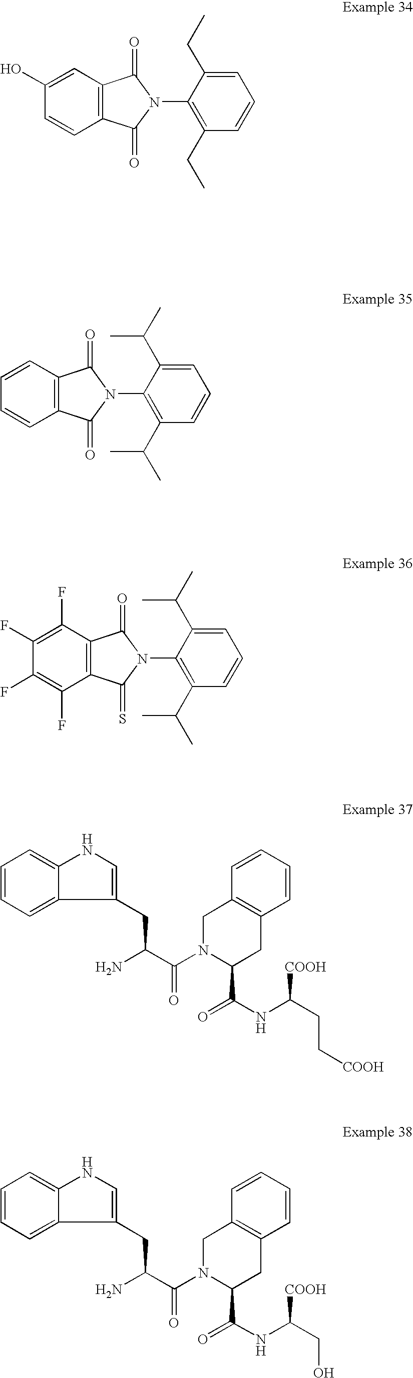

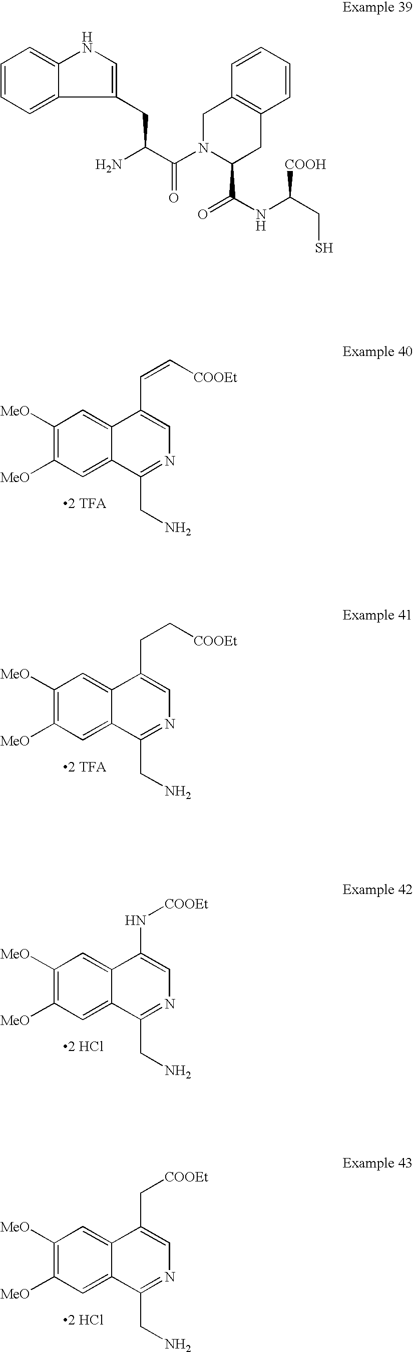

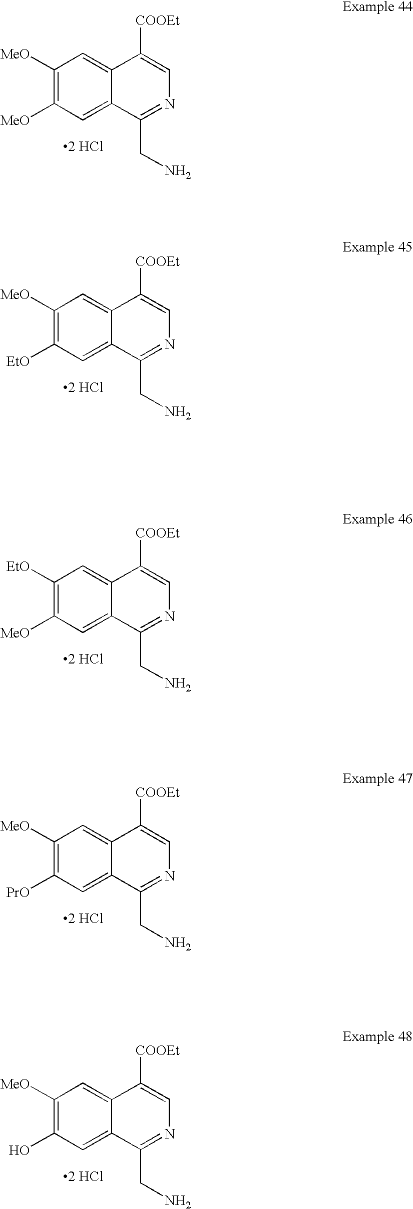

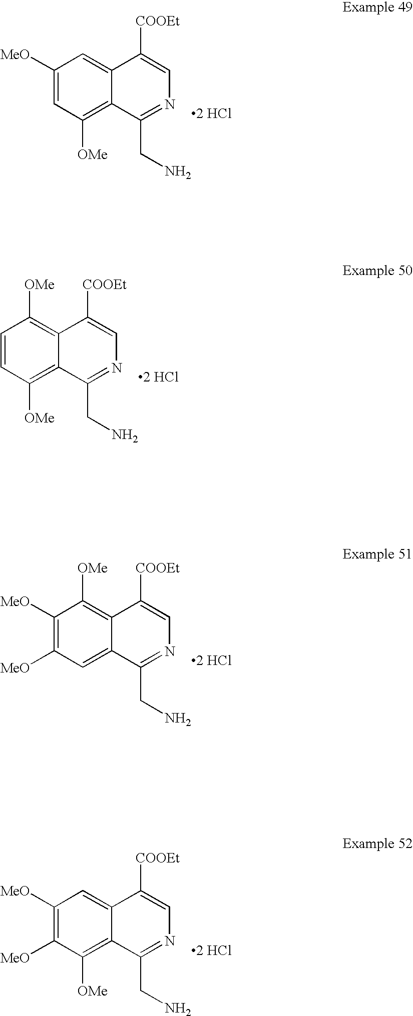

### Example 53

(S,S) 1-(2-Amino-propionyl)-2,5-dihydro-1H-pyrrole-2-carbonitrile-Example

### Example 54

(S,S) 1-(2-Amino-butyryl)-2,5-dihydro-1H-pyrrole-2-carbonitrile;

### Example 55

(S,S) 1 (2-Amino-3-methyl-butyryl)-2,5-dihydro-1H-pyrrole-2-carbonitrile;

### Example 56

(S,S) 1-(2-Amino-3,3-dimethyl-butyryl)-2,5-dihydro-1H-pyrrole-2-carbonitrile;

### Example 57

(S,S) 1-(2-Amino-4-methyl-pent-4-enoyl)-2,5-dihydro-1H-pyrrole-2-carbonitrile;

### Example 58

(S,S) 1-(2-Amino-3,3-diethyl-pentanoyl)-2,5-dihydro-1H-pyrrole-2-carbonitrile;

### Example 59

(S,S) 1-(2-Amino-2-cyclopentylacetyl)-2,5-dihydro-1H-pyrrole-2-carbo-nitrile;

### Example 60

(S,S) 1-(2-Amino-2-cyclohexylacetyl)-2,5-dihydro-1H-pyrrole-2-carbo-nitrile;

### Example 61

(S,S) 1-(2-Amino-2-cycloheptylacetyl)-2,5-dihydro-1H-pyrrole-2-carbo-nitrile;

### Example 62

(S,S) I-(2-Amino-2-bicyclo[2.2.2]oct-1-yl-acetyl)-2,5-dihydro-1H-pyrrole-2-carbonitrile;

### Example 63

(S,S) 1-(2-Adamantan-1-yl-2-amino-acetyl)-2,5-dihydro-1H-pyrrole-2-carbonitrile;

### Example 64

(S,S) 1-(2-Amino-2-phenylacetyl)-2,5-dihydro-1H-pyrrole-2-carbo-nitrile;

### Example 65

(S,S) 1-(2-Amino-2-(2,6 dimethylphenyl)acetyl)-2,5-dihydro-1H-pyrrole-2-carbonitrile;

### Example 66

(S,S) 1-(2-Amino-3,3-diphenyl-propionyl)-2,5-dihydro-1H-pyrrole-2-carbonitrile;

### Example 67

(S,S) 1-(2-Amino-(3(R)-methylpentanoyl)-2,5-dihydro-1H-pyrrole-2-carbonitrile;

### Example 68

(S,S) 1-(2-ino-(4-methylpentanoyl)-2,5-dihydro-1H-pyrrole-2-carbo-nitrile;

### Example 69

(S,S) 1-(2,6-Diamino-hexanoyl)-2,5-dihydro-1H-pyrrole-2-carbonitrile,

### Example 70

(S,S) 1-(2-Amino-6-dibenzylamino-hexanoyl)-2,5-dihydro-1H-pyrrole-2-carbonitrile;

### Example 71

(S,S) 1-(2-Amino-6-benzylamino-hexanoyl)-2,5-dihydro-1H-pyrrole-2-carbonitrile;

### Example 72

(S,S) [5-Amino-6-(2-cyano-2,5-dihydro-pyrrol-1-yl)-6-oxo-hexyl]-carbamic acid-tert-butyl ester;

### Example 73

(S,S) (5-Amino-6-(2-cyano-2,5-dihydro-pyrrol-1-yl)-6-oxo-hexyl]-carbamic acid 9-H-fluoren-9-ylmethyl ester;

### Example 74

(S,S) 4-Amino-5-(2-cyano-2,5-dihydro-pyrrol-1-yl)-5-oxo-pentanoic acid amide;

### Example 75

(S,S) 4-Amino-5-(2-cyano-2,5-dihydro-pyrrol-1-yl)-5-oxo-pentanoic acid benzylamide;

### Example 76

(S,S) 4-Amino-5-(2-cyano-2,5-dihydro-pyrrol-1-yl)-5-oxo-pentanoic acid benzyl ester;

### Example 77

(S,S) 4-Amino-5-(2-cyano-2,5-dihydro-pyrrol-1-yl)-5-oxo-pentanoic acid-tert-butyl ester;

### Example 78

(S,S) 1-(2-Amino-3-benzyloxy-propionyl)-2,5-dihydro-1H-pyrrole-2-carbonitrile;

### Example 79

(S,S) 1-(2-Amino-(4-methylsulfanyl-butyryl)-2,5-dihydro-1H-pyrrole-2-carbonitrile;

### Example 80

(S,S) 1-(2-Amino-(3-phenylpropionyl)-2,5-dihydro-1H-pyrrole-2-carbo-nitrile;

### Example 81

(S,S) 1-(Pyrrolidine-2-carbonyl)-2,5-dihydro-1H-pyrrole-2-carbonitrile;

### Example 82

(S,S) 6-{2-[2-(2-Cyano-2,5-dihydro-pyrrol-1-yl)-2-oxo-ethylamino]-ethylamino}-nicotinonitrile;

### Example 83

(S,S) 1-{2-[2-(5-Chloro-pyridin-2-ylamino)-ethylamino]-acetyl}-2,5-dihydro-1H-pyrrole-2-carbonitrile;

### Example 84

(S,S) 1-{2-[2-(5-Trifluoromethyl-pyridin-2-ylamino)-ethylamino]-acetyl}-2,5-dihydro-1H-pyrrole-2-carbonitrile;

### Example 85

(S,S) 1-[2-(1-Hydroxymethyl-cyclopentylamino)-acetyl]-2,5-dihydro-1-H-pyrrole-2-carbonitrile;

### Example 86

(S,S) 1-{2-[2-(5-Nitro-pyridin-2-ylamino)-ethylamino]-acetyl}-2,5-dihydro-1H-pyrrole-2-carbonitrile;

### Example 87

(S,S) 1-[2-(3-Isopropoxy-propylamino)-acetyl]-2,5-dihydro-1H-pyrrole-2-carbonitrile;

### Example 88

1-(Piperidine-3-carbonyl)-2,5-dihydro-1-H-pyrrole-2-S-carbonitrile;

### Example 89

1-(cis(2-Amino-cyclopenanecarbonyl))-2,5-dihydro-1-H-pyrrole-2-S-carbonitrile;

### Example 90

1-(3-R-Amino-5-phenyl-pentanoyl)-2,5-dihydro-1-H-pyrrole-2-S-carbo-nitrile;

### Example 91

1-(3-S-Amino-5-phenyl-pentanoyl)-2,5-dihydro-1-H-pyrrole-2-S-carbo-nitrile;

### Example 92

1-(3-S-Amino-4-phenyl-butyryl)-2,5-dihydro-1-H-pyrrole-2-S-carbo-nitrile;

### Example 93

1-(3-R-Amino-3-phenyl-propionyl)-2,5-dihydro-1-H-pyrrole-2-S-carbo-nitrile;

### Example 94

1-(Morpholine-2-carbonyl)-2,5-dihydro-1-H-pyrrole-2-S-carbonitrile;

### Example 95

1-(3-R-Amino-6-phenyl-hex-5-enoyl)-2,5-dihydro-1-H-pyrrole-2-S-carbonitrile;

### Example 96

1-(3-R-Amino-4-benzo[b]thiophen-2-yl-butyryl)-2,5-dihydro-1-H-pyrrole-2-S-carbonitrile;

### Example 97

1-(3-R-amino-4-pyridin-3-yl-butyryl)-2,5-dihydro-1-H-pyrrole-2-S-carbonitrile;

### Example 98

1-[3-S-Amino-4-(4-benzyloxy-phenyl)-butyryl]-2,5-dihydro-1-H-pyrrole-2-S-carbonitrile;

### Example 99

1-[2-S-Pyrolidin-2-yl-acetyl)-2,5-dihydro-1-H-pyrrole-2-S-carbonitrile;

### Example 100

1-[4-(2-Chloro-phenyl)-pyrrolidine-3-carbonyl]-2,5-dihydro-1-H-pyrrole-2-S-carbonitrile;

### Example 101

1-(4-R-Phenyl-pyrrolidine-3-S-carbonyl)-2,5-dihydro-1-H-pyrrole-2-S-carbonitrile;

### Example 102

N-(Cyclopentylglycyl) pyrrolidine;

### Example 103

N-(L-Cyclohexylglycyl) pyrrolidine;

### Example 104

N-(L-Cyclohex-3-enylglycyl) pyrrolidine;

### Example 105

N-(cis-2-Aminocyclohexylcarbonyl) pyrrolidine;

### Example 106

N-(trans-2-Aminocyclohexylcarbonyl) pyrrolidine;

### Example 107

N-(trans-2-Aminocyclohex-4-enylcarbonyl) pyrrolidine;

### Example 108

N-(trans-2-Aminocyclopentylcarbonyl) pyrrolidine;

### Example 109

### N-(trans-2-Aminocyclooctylcarbonyl) pyrrolidine;

### Example 110

L-Isoleucyl-L-prolinenitrile;

### Example 111

L-(N-Benzyloxycarbonyllysyl)-L-prolinenitrile;

### Example 112

L-Prolyl-L-prolinenitrile;

### Example 113

L-4-Thiaprolyl-L-prolinenitrile;

### Example 114

3-Thiaprolyl-L-prolinenitrile;

### Example 115

L-Cyclohexylglycyl-L-prolinenitrile;

### Example 116

L-Cyclopentylglycyl-L-prolinenitrile;

### Example 117

L-tert-Butylglycyl-L-prolinenitrile;

### Example 118

L-Isoleucyl-L-4-thiaprolinenitrile;

### Example 119

L-Isoleucyl-3-thiaprolinenitrile;

### Example 120

L-Cyclohexylglycyl-L-4-thiaprolinenitrile;

### Example 121

L-(N-Benzyloxycarbonyllysyl)-L-4-thiaprolinenitrile;

### Example 122

L-Isoleucyl-L-4-oxaprolinenitrile;

### Example 123

### N-(L-Isoleucyl)picolinonitrile;

### Example 124

### N-(Lisoleucyl)-5-thiapicolinonitrile;

### Example 125

L-Isoleucyl-L-4-thiaprolinenitrile-S,S-dioxide;

### Example 126

L-Isoleucyl-L-4-thiaprolinenitrile-S-oxide;

### Example 127

N-(1S, 2S-2-Aminocyclohexylcarbonyl)-L-prolinenitrile;

### Example 128

N-(1R, 2R-2-Aminocyclohexylcarbonyl)-L-prolinenitrile;

### Example 129

N-(1S, 2S-2-Aminocyclopentylcarbonyl)-L-prolinenitrile;

### Example 130

N-(1R, 2R-2-Aminocyclopentylcarbonyl)-L-prolinenitrile;

### Example 131

N-(1S, 2S-2-Aminocyclohex-4-enylcarbonyl)-L-prolinenitrile;

### Example 132

N-(1R, 2R-2-Aminocyclohex-4-enylcarbonyl)-L-prolinenitrile;

### Example 133

N-(1S, 2R-2-Aminocyclohexylcarbonyl)-L-prolinenitrile;

### Example 134

N-(1S, 2R-2-Aminocyclohex-4-enylcarbonyl)-L-prolinenitrile;

### Example 135

N-(trans-2-Aminocyclohexylcarbonyl)-L-prolinal;

### Example 136

N-(1S, 2S-2-Aminocyclopentylcarbonyl)-L-prolinal;

### Example 137

N-(1R, 2R-2-Aminocyclopentylcarbonyl)-L-prolinal;

### Example 138

N-(trans-2-Aminocyclopentylcarbonyl) pyrrolidine-2-boronic acid;

### Example 139

N-(1S, 2S-2-Aminocyclohexylcarbonyl) pyrrolidine-2-boronic acid;

### Example 140

N-(1R,2R-2-Aminocyclohexylcarbonyl) pyrrolidine-2-boronic acid;

### Example 141

N-(1S, 2S-2-Aminocyclohex-4-enylcarbonyl) pyrrolidine-2-boronic acid;

### Example 142

N-(1R,2R-2-Aminocyclohex-4-enylcarbonyl)pyrrolidine-2-boronic acid;

### Example 143

N—(N^{ω}-Benzyloxycarbonylmethyl)asparaginyl)pyrrolidine;

### Example 144

N—(N^{ω}-(Carboxymethyl)asparaginyl)pyrrolidine;

### Example 145

N—(N^{ω}-(3-Carboxypropyl)asparaginyl)pyrrolidine;

### Example 146

N—(N^{ω}-(2-(Benzyloxycarbonyl)ethyl)asparaginyl)pyrrolidine;

### Example 147

N—(N^{ω}-(2-Carboxyethyl)asparaginyl)pyrrolidine;

### Example 148

N—(N^{ω}-(5-Benzyloxycarbonyl)pentyl)asparaginyl)pyrrolidine;

### Example 149

N—(N^{ω}-(5-Carboxypentyl)asparaginyl)pyrrolidine;

### Example 150

N—(N^{ω}-(3-(Benzyloxycarbonyl)propyl)asparaginyl)pyrrolidine;

### Example 151

N—(N^{ω}-(Benzyloxycarbonylmethyl)glutaminyl)pyrrolidine;

### Example 152

N—(N^{ω}-(Carboxymethyl)glutaminyl)pyrrolidine;

### Example 153

N—(N^{ω}-(2-(Benzyloxycarbonyl)ethyl)glutaminyl)pyrrolidine;

### Example 154

N—(N^{ω}-(3-(Benzyloxycarbonyl)propyl)glutaminyl)pyrrolidine;

### Example 155

N—(N^{ω}-(3-Carboxypropyl)glutaminyl)pyrrolidine;

### Example 156

N—(N^{ω}-(5-(Benzyloxycarbonyl)pentyl)glutaminyl)pyrrolidine;

### Example 157

N—(N^{ω}-(5-Carboxypentyl)glutaminyl)pyrrolidine;

### Example 158

N—(N^{ω}-(2-Carboxyethyl)glutaminyl)pyrrolidine;

### Example 159

N—(N^{ω}-(7-(Benzyloxycarbonyl)heptyl)glutaminyl)pyrrolidine;

### Example 160

N—(N^{ω}-(7-Carboxyheptyl)glutaminyl)pyrrolidine;

### Example 161

N—(N^{ω}-(7-(3-(Benzyloxycarbonylamino)propylaminocarbonyl)heptyl) glutaminyl)pyrrolidine;

### Example 162

N—(N^{ω}-(6-(5-(Benzyloxycarbonyl)pentylaminocarbonyl)hexyl) glutaminyl)pyrrolidine;

### Example 163

N—(N^{ω}-(6-(5-Carboxypentylaminocarbonyl)hexyl)glutaminyl) pyrrolidine;

### Example 164

N—(N^{ω}-(7-(3-Aminopropylaminocarbonyl)heptyl)glutaminyl) pyrrolidine;

### Example 165

N—(N^{ω}-(11-(Benzyloxycarbonyl)undecyl)glutaminyl)pyrrolidine;

### Example 166

N—(N^{ω}-(11-Carboxyundecyl)glutaminyl)pyrrolidine;

### Example 167

N—(N^{ω}-(6-(Benzyloxycarbonyl)hexyl)glutaminyl)pyrrolidine;

### Example 168

N—(N^{ω}-(6-Carboxyhexyl)glutaminyl)pyrrolidine;

### Example 169

N—(N^{ω}-(5-(2,2,2-Trifluoroethylaminocarbonyl)pentyl)glutaminyl) pyrrolidine;

### Example 170

N—(N^{ω}-(5-(2,2,3,3,4,4,4-Heptafluorobutylaminocarbonyl)pentyl) glutaminyl)pyrrolidine;

### Example 171

N—(N^{ω}-(5-(6-Hydroxyhexylaminocarbonyl)pentyl)glutaminyl) pyrrolidine;

### Example 172

N—(N^{ω}-(5-(3-Phenylpropylaminocarbonyl)pentyl)glutaminyl) pyrrolidine;

### Example 173

N—(N^{ω}-(5-(4-Phenylbutylaminocarbonyl)pentyl)glutaminyl) pyrrolidine;

### Example 174

N—(N^{ω}-(5-(Dibutylaminocarbonyl)pentyl)glutaminyl)pyrrolidine;

### Example 175

N—(N^{ω}-(5-(Dihexylaminocarbonyl)pentyl)glutaminyl)pyrrolidine;

### Example 176

N—(N^{ω}-(5-(Benzylaminocarbonyl)pentyl)glutaminyl)pyrrolidine;

### Example 177

N—(N^{ω}-(4-(Benzyloxycarbonyl)butyl)glutaminyl)pyrrolidine;

### Example 178

N—(N^{ω}-(4-Carboxybutyl)glutaminyl)pyrrolidine;

### Example 179

N—(N^{ω}-(5-(Ethylaminocarbonyl)pentyl)glutaminyl)pyrrolidine;

### Example 180

N—(N^{ω}-(6-Hydroxyhexyl)glutaminyl)pyrrolidine;

### Example 181

N—(N^{ω}-(5-(Piperidine-1-carbonyl)pentyl)glutaminyl)pyrrolidine;

### Example 182

N—(N^{ω}-(5-Carbamoylpentyl)glutaminyl)pyrrolidine;

### Example 183

N—(N^{ω}-(5-(Decylaminocarbonyl)pentyl)glutaminyl)pyrrolidine;

### Example 184

N—(N^{ω}-(5-(Heptylaminocarbonyl)pentyl)glutaminyl)pyrrolidine;

### Example 185

N—(N^{ω}-(5-(Cyclohexylmethylaminocarbonyl)pentyl)glutaminyl) pyrrolidine;

### Example 186

N—(N^{ω}-(5-(3-(Benzyloxycarbonylamino)propylaminocarbonyl)pentyl) glutaminyl)pyrrolidine;

### Example 187

N—(N^{ω}-(5-(3-Aminopropylaminocarbonyl)pentyl)glutaminyl) pyrrolidine;

### Example 188

N—(N^{ω}-(5-(3-Guanidinopropylaminocarbonyl)pentyl)glutaminyl) pyrrolidine;

### Example 189

N—(N^{ω}-(5-(4-Sulfoxyphenylaminocarbonyl)pentyl)glutaminyl) pyrrolidine;

### Example 190

N—N^{ω}-(5-(1-Benzylpiperidin-4-ylaminocarbonyl)pentyl)glutaminyl) pyrrolidine;

### Example 191

N—(N^{ω}-(5-(Piperidin-4-ylaminocarbonyl)pentyl)glutaminyl)pyrrolidine;

### Example 192

N—(N^{ω}-(4-(N-Benzyloxycarbonyl-N-(3-benzyloxycarbonyl-aminopropyl)-aminocarbonyl)butyl)glutaminyl)pyrrolidine;

### Example 193

N—(N^{ω}-(4-(3-Aminopropylaminocarbonyl)butyl)glutaminyl) pyrrolidine;

### Example 194

N—(N^{ω}-(5-(Benzyloxycarbonyl)pentyl)glutaminyl)prolinenitrile;

### Example 195

N—(N^{ω}-(6-(5-(Benzyloxycarbonyl)pentylaminocarbonyl)hexyl) homoglutaminyl)-pyrrolidine;

### Example 196

N—(N^{ω}-(6-(5-Carboxypentylaminocarbonyl)hexyl)homoglutaminyl) pyrrolidine;

### Example 197

N—(N^{ω}-(5-(Benzyloxycarbonyl)pentyl)homoglutaminyl)pyrrolidine;

### Example 198

N—(N^{ω}-(5-Carboxypentyl)homoglutaminyl)pyrrolidine;

### Example 199

### (3S)-3-Amino-N-(5-carboxypentyl)4-oxo4-(1-pyrrolidino) butanesulfonamide;

### Example 200

N—(N^{ω}-(8-(Glucosaminothiocarbonylamino)octyl)glutninyl)pyrrolidine;

### Example 201

N-((2S)-2-Amino-3-(7-carboxyheptanoylamino)propanoyl)pyrrolidine;

### Example 202

N-((2S)-2-Amino-3-(7-(benzyloxycarbonyl)heptanoylamino) propanoyl)pyrrolidine;

### Example 203

N—(N^{ω}-(5-Carboxypentanoyl)ornithinyl)pyrrolidine;

### Example 204

N—(N^{ω}-(5-(Methyloxycarbonyl)pentanoyl)ornithinyl)pyrrolidine;

### Example 205

N—(N^{ω}-(6-Aminohexanoyl)lysinyl)pyrrolidine;

### Example 206

N—N^{ω}-(4-Aminobutanoyl)lysinyl)pyrrolidine;

### Example 207

N—(N^{ω}-(4-(Pentafluorobenzenesulfonylamino)butanoyl)lysinyl) pyrrolidine;

### Example 208

N—(N^{ω}-(4-(Pentafluorobenzoylamino)butanoyl)lysinyl)pyrrolidine;

### Example 209

N—(N^{ω}-(4-(2,2,2-Trifluoroethanesulfonylamino)butanoyl)lysinyl) pyrrolidine;

### Example 210

N—(N^{ω}-(12-(7-Benzyloxycarbonylamino)heptanoylamino)dodecanoyl) lysinyl)pyrrolidine;

### Example 211

N—(N^{ω}-(12-(7-Aminoheptanoylamino)dodecanoyl)lysinyl)pyrrolidine;

### Example 212

N—(N^{ω}-(6-(6-(6-(Benzyloxycarbonylamino(hexanoylamino) hexanoylamino)hexanoyl)lysinyl)pyrrolidine;

### Example 213

N—(N^{ω}-(6-(6-(6-Aminohexanoylamino)hexanoylamino)hexanoyl) lysinyl)pyrrolidine;

### Example 214

N—(N^{ω}-(4-Carboxybutanoyl)lysinyl)pyrrolidine;

### Example 215

N—(N^{ω}-(4-(Benzyloxycarbonyl)butanoyl)lysinyl)pyrrolidine;

### Example 216

N—(N^{ω}-(7-Aminoheptanoyl)lysinyl)pyrrolidine;

### Example 217

N—(N^{ω}-(8-Aminooctanoyl)lysinyl)pyrrolidine;

### Example 218

N—(N^{ω}-Octadecanoyllysinyl)pyrrolidine;

### Example 219

N—(N^{ω}-(7-Guanidinoheptanoyl)lysinyl)pyrrolidine;

### Example 220

N—(N^{ω}-Octanesulfonyllysinyl)pyrroiidine;

### Example 221

N—(N^{ω}-(12-Aminododecanoyl)lysinyl)pyrrolidine;

### Example 222

N—(N^{ω}-(2-(Benzyloxycarbonylamino)ethanoyl)lysinyl)pyrrolidine;

### Example 223

N—(N^{ω}-(3-(Benzyloxycarbonylamino)propanoyl)lysinyl)pyrrolidine;

### Example 224

N—(N^{ω}-(4(Benzyloxycarbonylamino)butanoyl)lysinyl)pyrrolidine;

### Example 225

N—(N^{ω}-(3-Aminopropanoyl)lysinyl)pyrrolidine;

### Example 226

N—(N^{ω}-(6-(Benzyloxycarbonylamino)hexanoyl)lysinyl)pyrrolidine;

### Example 227

N—(N^{ω}-(2-Guanidinoethanoyl)lysinyl)pyrrolidine;

### Example 228

N—(N^{ω}-(3-Aminopropanoyl)lysinyl)pyrrolidine;

### Example 229

N—(N^{ω}-(3-Guanidinopropanoyl)lysinyl)pyrrolidine;

### Example 230

N—N^{ω}-(4-Guanidinobutanoyl)lysinyl)pyrrolidine;

### Example 231

N—(N^{ω}-(6-Guanidinohexanoyl)lysinyl)pyrrolidine;

### Example 232

N—(N^{ω}-(7-Aminoheptanoyl)lysinyl)prolinenitrile;

### Example 233

N—(N^{ω}-(8-Aminooctanoyl)lysinyl)prolinenitrile;

### Example 234

N—(O-(2-(5-Carboxypentylamino)-2-oxoethyl)serinyl)pyrrolidine;

### Example 235

N—(O-(2-(5-(Benzyloxycarbonyl)pentylamino)-2-oxoethyl)serinyl) pyrrolidine;

### Example 236

N—(O-(2-(4-(Benzyloxycarbonyl)butylamino)-2-oxoethyl)serinyl) pyrrolidine;

### Example 237

N—(O-(2-(4-Carboxybutylamino)-2-oxoethyl)serinyl)pyrrolidine;

### Example 238

N—(O-Methylthreoninyl)pyrrolidine;

### Example 239

N—(O-Ethylthreoninyl)pyrrolidine;

### Example 240

N—(O-Hexylthreoninyl)pyrrolidine;

### Example 241

N—(O-(2-(5-(Benzyloxycarbonyl)pentylamino)-2-oxoethyl)threoninyl) pyrrolidine;

### Example 242

N—(O-(2-(5-Carboxypentylamino)-2-oxoethyl)threoninyl)pyrrolidine;

### Example 243

N—(O-(2-(4(Benzyloxycarbonyl)butylamino)-2-oxoethyl)threoninyl) pyrrolidine;

### Example 244

N—(O-(2-(4-Carboxybutylamino)-2-oxoethyl)threoninyl)pyrrolidine;

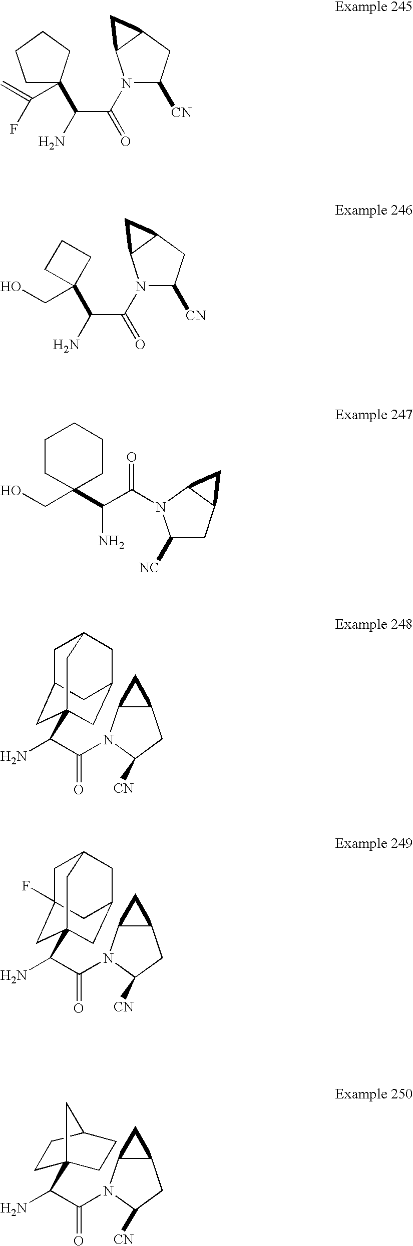

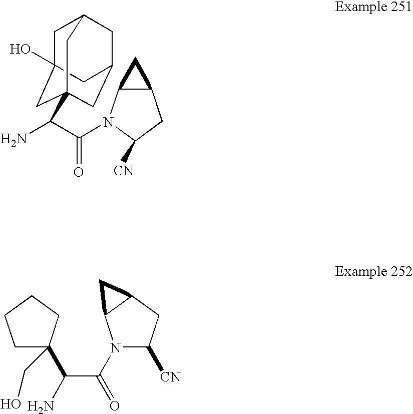

### Preparation of Compounds of the Invention

Preparation of Exemplary Compounds 21-30:

Compounds of Formula V can be prepared according to the general procedure described at Cols. 2-4 in U.S. Pat. No. 6,172,081, which procedure is herein incorporated by reference. The specific EXAMPLES 21-30 can be prepared by the methods described at Cols. 7-17 in U.S. Pat. No. 6,172,081, which methods are herein incorporated by reference.

Preparation of Exemplary Compounds 31-36:

The n-substituted phthalimides of Formula VI can be prepared via conventional reaction of the corresponding phthalic anhydrides with substituted anilines in acetic acid upon reflux. On cooling, precipitated products are filtered and recrystallized from ethanol (see: Vogel's Textbook of Practical Organic Chemistry, 5th Edition; Longman, Singapore: 1996, p. 1276). Furthermore, preparation of aminosubstituted phthalimides (see, e.g., EXAMPLE 31) can be accomplished by catalytic (palladium on carbon) hydrogenation of the corresponding nitro compounds according to procedures which are themselves well-known in the art (see, e.g., the procedure described in Bailleux, V.; Vallee, L.; Nuyts, J.-P.; Vamecq, J.; Chem. Pharm. Bull., 1994, v. 42, No. 9, pp. 1817-1821).

Preparation of Exemplary Compounds 40-52:

The compounds of Formula VII can be prepared from commercially available starting materials according to procedures which are themselves well-established in the art (see Coppola, G. M., Zhang, L. Y., Schuster, H. F., Russell M. E., Hughes, T. E., Bioorg. Med. Chem. Lett. 10 (2000) 1555-1558).

Preparation of Exemplary Compounds 53-101:

The compounds of Formula IX, and pharmaceutically acceptable salts thereof, can be prepared according to the general methods disclosed at paragraphs 0150-0158 of United States Patent Application Publication 2001/0031780 A1, which methods are herein incorporated by reference.

Preparation of Exemplary Compounds 102-244:

The compounds of Formulae Xa-Xf can be prepared by the synthetic methods taught at Cols. 27-42 of U.S. Pat. No. 5,939,560, which methods are herein incorporated by reference.

Preparation of Exemplary Compounds 245-252:

The compounds of Formula XI can be prepared by the general methods taught at Cols. 6-15, and by the specific methods described at Cols. 25-88, of U.S. Pat. No. 6,395,767, which methods are herein incorporated by reference.

Preparation of Exemplary Compounds 3, 13, and 14:

The preparation of 2-cyano-fluoro-1-cyclohexylglycylpyrrolidine hydrogen chloride (9, EXAMPLE 14), 2-cyano-4-fluoro-1-(octahydroindole-2-carbonyl)-pyrrolidine hydrogen chloride (10, EXAMPLE 3), and 2-cyano-4-fluoro-1-(sec-butyl)glycylpyrrolidine hydrogen chloride (11, EXAMPLE 13) was conducted according to the following Scheme 1: 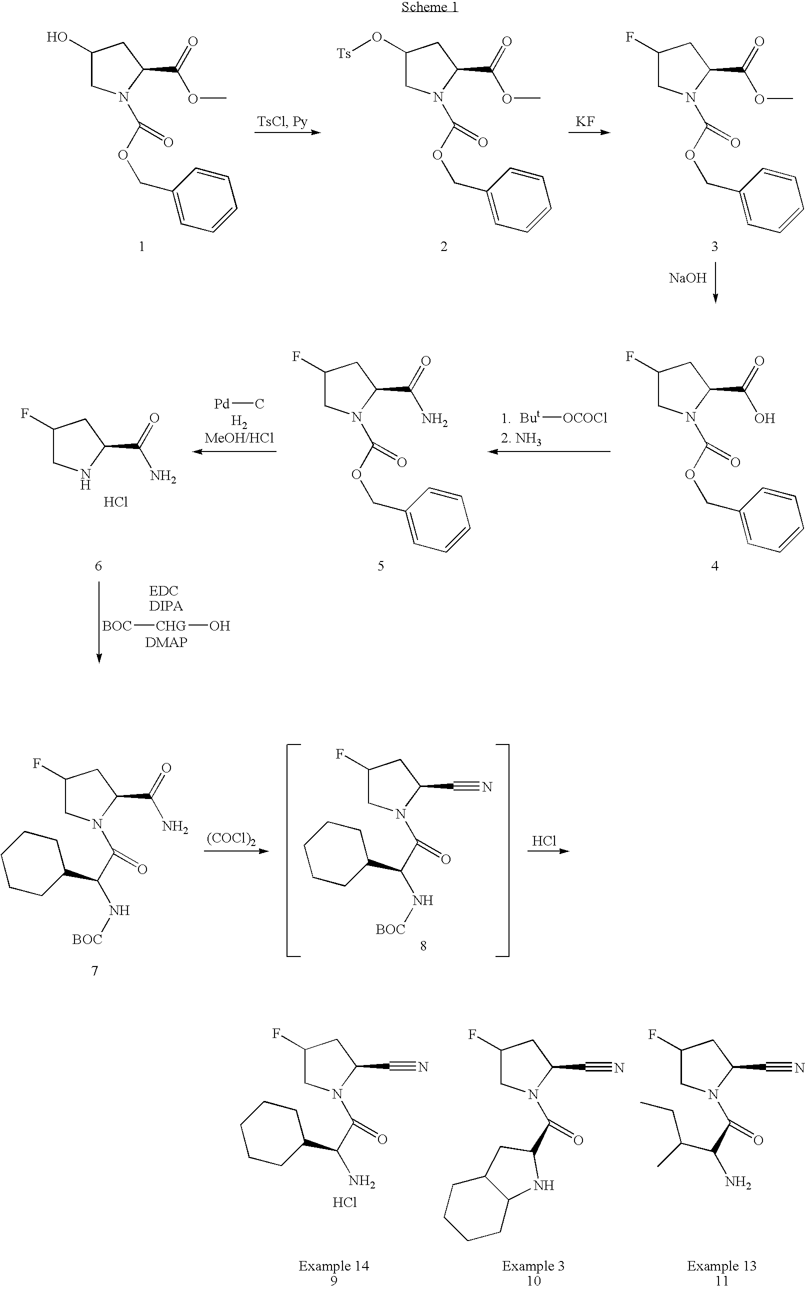
N-benzyloxycarbonyl-O-p-tolysulfonylhydroxy-L-proline methyl ester (2). To a solution of N-benzyloxycarbonyl-hydroxy-L-proline methyl ester (1, 25.0 g, 0.09 mol) in dry pyridine (60 mL) was added a solution of p-toluenesulfonyl chloride (21.0 g, 0.11 mol) in dry pyridine (20 mL) at 0° C. with stirring under nitrogen. The resulting mixture was left stirring for 3 days at the same temperature. At the end of this time, ice-cold HCl solution (2N, 300 mL) was added to the mixture; the whole was stirred for 5 min, and then extracted with ethyl acetate (3×300 mL). The organic layer was separated, dried over anhydrous sodium sulfate, filtered, and the solvent was removed in vacuum to give an oil, which was purified by flash column chromatography (silica gel; eluent: ethyl acetate:hexanes, 1:1). Yellowish oil, yield: 34.5 g.
Anal. Cacld for C₂₁H₂₃NSO₇: C, 58.19; H, 5.35; N, 3.23. Found: C, 58.14; H, 5.18; N, 3.20.
MS:434 [M^{+].}
N-benzyloxycarbonyl-4-fluoro-L-proline methyl ester (3). To a stirred solution of N-benzyloxycarbonyl-O-p-tolysulfonylhydroxy-L-proline methyl ester (2, 13.4 g, 0.031 mol) in diethylene glycol (125 mL) was added potassium fluoride (12.4 g, 0.21 mol). Resulting mixture was heated under nitrogen at 80° C. overnight; water (100 mL) was added, and the mixture was extracted with ethyl acetate (2×200 mL). The organic layer was washed with brine (50 mL), separated, dried over anhydrous sodium sulfate, and filtered. Solvents were removed in vacuum to give oil, which then was purified by flash column chromatography (silica gel; eluent: ethyl acetate:hexanes, 2:1). Colorless oil, yield: 5.5 g.
N-benzyloxycarbonyl 4-fluoro-L-proline (4). To a solution of N-benzyloxycarbonyl-4-fluoro-L-proline methyl ester (3, 5.5 g, 0.02 mol) in methanol (35 mL) was added sodium hydroxide solution (2N, 15 mL) at 0-5° C. and stirring. The temperature of the reaction mixture was allowed to rise to 20° C. during overnight stirring. After completion of hydrolysis, water (50 mL) was added, and the whole was extracted with ether (2×50 mL). Aqueous layer was separated, and acidified with HCl solution (6N) to pH 1. It was extracted with ethyl acetate (2×100 mL); organic layer was separated, dried over anhydrous sodium sulfate, filtered, and concentrated in vacuum. Yellowish oil, yield: 2.5 g. Anal. Cacld. for C₁₃H₁₄FNO₄: C, 58.42; H, 5.28; N, 5.24. Found: C, 58.53; H, 5.45; N, 5.14.
MS: 266 [M^{-].}
N-benzyloxycarbonyl-4-fluoro-L-prolinamide (5). To a stirred solution of N-benzyloxycarbonyl-4-fluoro-L-proline (4, 2.37 g, 8.9 mmol) and triethylamine (1.5 mL) in THF (50 mL) was added isobutyl chloroformate (1.38 mL, 11 mmol) at 0° C. under nitrogen. Resulting mixture was stirred at this temperature for 40 min, then ammonia solution in methanol (2M, 20 mL) was added portionwise within 20 min. After stirring for additional 4 hours, the mixture was poured onto water (100 mL). The whole was extracted with ethyl acetate (3×100 mL), organic layer was separated, dried over anhydrous sodium sulfate, filtered, and concentrated in vacuum to give an oil, which was purified by flash column chromatography (silica gel; eluent: ethyl acetate:MeOH, 4:1). Yield: 2.0 g.
Anal. Cacld. for C₁₃H₁₅F₂NO₃: C, 58.64; H, 5.68; N, 10.52. Found: C, 58.92; H, 5.85; N, 10.09.
MS: 267 [M^{+].}
4-Fluoro-L-prolinamide hydrogen chloride (6). A mixture containing N-benzyloxycarbonyl-4-fluoro-L-prolinamide (5, 0.9 g, 3.4 mmol), Pd—C (10%, 0.8 g), HCl solution in dioxane (4M, 2.5 mL), and methanol (15 mL) was hydrogenated at 45 psi at room temperature for 1 h. Reaction mixture was filtered through Celite plug, and solvents removed in vacuum to give off-white solid; yield: 0.45 g.
MS: 133 [M^{+].}
2-Carbamoyl-4-fluoro-1-N-t-butloxycarbonyl-cyclohexylglycylpyrrolidine (7). To a stirred mixture of 4-fluoro-L-prolinamide hydrogen chloride (6, 0.4 g, 2.38 mmol), 4-(N,N-dimethylamino)pyridine (50 mg), diisopropylethylamine (1.2 mL), and BOC-CHG-OH (0.72 g, 2.8 mmol) in dichloromethane (4 mL) was added 1-(3-dimethylaminopropyl)-3-ethylcarbodiimide hydrochloride (EDC, 0.62 g, 3.2 mmol) in one portion at room temperature. The resulting mixture was stirred overnight at room temperature. Solvent was removed in vacuum; water (30 mL) and ethyl acetate (50 mL) were added to the mixture. After extraction, the organic layer was separated, washed with water (15 mL), separated, and dried over anhydrous sodium sulfate. Removal of the solvent in vacuum gave an oil, which was purified by flash column chromatography (silica gel; eluent: ethyl acetate:TBF, 4:1). Yield: 0.510 g.
MS:372 [M^{+].}
2-Cyano-4-fluoro-1-N-t-butyloxycarbonyl-cyclohexylglycylpyrrolidine (8). Oxalyl chloride (0.14 mL, 1.6 mmol) was added dropwise to a stirred mixture of acetonitrile (2 mL) and DMF (0.135 mL) at 0-5° C. After stirring at this temperature for 40 minutes under nitrogen, a solution of 2-carbamoyl-4-fluoro-1-N-t-butyloxycarbonyl-cyclohexylglycylpyrrolidine (7, 0.500 g, 1.34 mmol) in acetonitrile (2 mL) was added slowly, and reaction mixture was stirred for additional 1 h. Triethylamine (5 mL) was added to quench the reaction, and the whole was stirred for 15 min. Solids were removed by filtration, and solvents were removed in vacuum to give an oil, which was purified by flash column chromatography (silica gel; eluent: ethyl acetate:hexanes, 1:1). Off-white wax; yield: 0.215 g.
Anal. Calcd. for C₁₈H₂₈N₃O₃F: C, 61.17; H, 7.99; N, 11.89. Found: C, 61.42; H, 8.14; N, 11.82.
MS: 354 [M^{+].}
2-Cyano-4-fluoro-1-cyclohexylglycylpyrrolidine hydrogen chloride (9, EXAMPLE 14). 2-Cyano-4-fluoro-1-N-t-butyloxycarbonyl-cyclohexylglycylpyrrolidine (8, 0.2 g, 0.6 mmol) was added in one portion to a stirred solution of HCl in diethyl ether (2M, 24 mL) under nitrogen. The resulting mixture was stirred at room temperature for 4 h. After the completion of the reaction, the liquid phase was decanted, and the solid was washed with dry diethyl ether (3×1 mL), and then dried in vacuum. White solid; yield: 40 mg (24%).
Anal. Calcd. for C₁₃H₂₁ClFN₃OH₂O: C, 50.73; H, 7.53; N, 13.65. Found: C, 50.98; H, 7.38; N, 13.63.
NMR (300 MHz, δ, D₂O): 5.56 (s, 0.5H), 5.38 (s, 0.5H), 5.02 (d, J=9 Hz, 11), 4.13-3.71 (m, 3H), 2.70-2.31 (m, 2H), 1.96-1.83 (m, 1H), 1.80-1.52 (m, 5H), 1.32-0.97 (m, 5H).
MS: 254[M^{+].}
2-Cyano-4-fluoro-1-(octahydro-indole-2-carbonyl-pyrrolidine hydrogen chloride (10, EXAMPLE 3). The procedure used for its preparation is analogous to that used for Example 14, except that during the coupling of compound 6 the BOC-OIC-OH was used instead of BOC-CHG-OH, to provide the required OIC moiety on the lower part of the molecule. White solid; yield: 40%.
NMR (300 MHz, δ, D₂O): 5.57 (s, 0.5H), 5.40 (s, 0.5H), 5.03 (d, J=9 Hz, 1H), 4.54-4.48 (m, 1H), 3.99-3.65 (m, 4H), 2.71-2.42 (m, 4H), 2.06-1.96 (m, 1H), 1.92-1.78 (m, 1H), 1.71-1.46 (m, 4H), 1.41-1.22 (m, 3H)
MS: 266 [M^{+].}
2-Cyano-4-fluoro-1-(sec-butyl)glycylpyrrolidine hydrogen chloride (11, EXAMPLE 13). The procedure used for its preparation is analogous to that used for Example 14, except that during the coupling of compound 6 the BOC-ILE-OH was used instead of BOC-CHG-OH, to provide the required Ile moiety. White solid; yield: 37%. NMR (300 MHz, δ, D₂O): 5.57 (s, 0.5H), 5.40 (s, 0.51), 5.03 (d, J=12 Hz), 4.14-4.72 (m, 3H), 2.71-2.34 (m, 2H), 2.03-1.90 (m, 1H), 1.53-1.90 (m, 21), 1.00 (d, J=6 Hz, 3H), 0.88-0.80 (m, 3H).
MS: 266 [M^{+].}

Preparation of Exemplary Compound 15:

The preparation of 1-[2-(adamant-1-ylamino)acetyl]4-fluoropyrrolidine-2-carbonitrile hydrochloride (17, EXAMPLE 15) was conducted according to Scheme 2, below.

Benzyl α-admantanaminoacetate (12). To a stirred solution of 1-adamantanamine (24.5 g, 0.16 mol) in dichloromethane (200 mL) was added a solution of benzyl α-bromoacetate (10.3 g, 0.045 mol) in dichloromethane (50 mL) dropwise under nitrogen at 0-5° C. Resulting mixture was stirred overnight at room temperature. After removal of solids by filtration, the solvent was evaporated to give yellowish oil, which was purified by flash column chromatography (silica gel; eluent: ethyl acetate:hexanes, 1:1). Clear oil; yield: 11.02 g (73%).

MS: 300 [M^{+].}

Benzyl N-BOC-α-admantanaminoacetate (13). To a stirred solution of benzyl α-admantanaminoacetate (12, 6.0 g, 0.02 mol) in dichloromethane (30 mL) was added BOC-anhydride (4.4 g, 0.02 mol) in dichloromethane (20 mL) dropwise under nitrogen at 0-5° C. Resulting mixture was stirred overnight at room temperature. After removal of solids by filtration, the solvent was evaporated to give yellowish oil, which was purified by flash column chromatography (silica gel; eluent: ethyl acetate:hexanes, 1:3). Colorless oil; yield: 5.11 g.
MS: 400 [M⁺]. 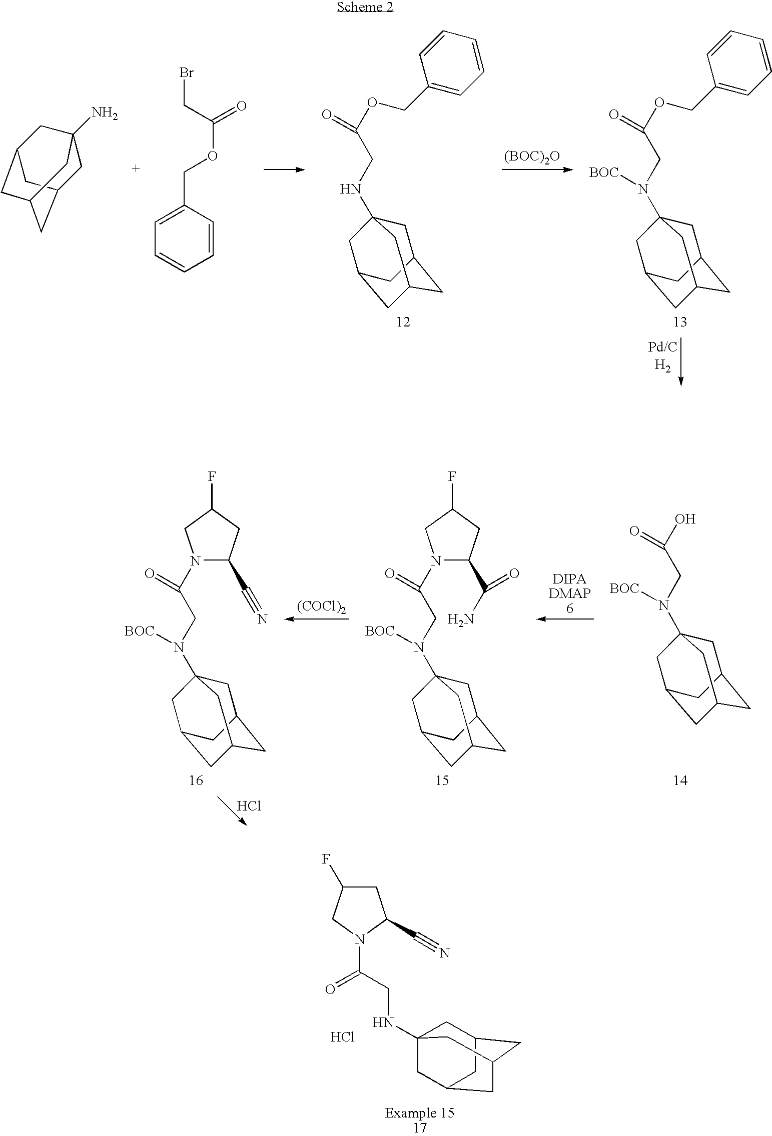
N-BOC-α-admantanaminoacetic acid (14). A mixture of Pd—C (10%, 0.3 g), benzyl N-BOC-α-admantanaminoacetate (13, 3.0 g, 0.0075 mol) in ethyl acetate (50 mL) was hydrogenated for 4 h at 45 psi and room temperature. Solids were filtered off through Celite plug, and solvent was removed in vacuum to give 14; yield 2.5 g.
MS: 308 [M^{+].}
N-BOC-1-[2-(Adamant-1-ylamino)acetyl]-4-fluoropyrrolidine-2-carboxamide (15), and N-BOC-1-2-(adamant-1-ylamino)acetyl]-4-fluoropyrrolidine-2-carbonitrile (16) were prepared analogously to compounds 7 and 8, of Scheme 1.
2-Cyano-4-fluoro-1-admantaneamino-pyrrolidine hydrogen chloride (17, EXAMPLE 15). The procedure of BOC protecting group removal is analogous to that used for Example 14. White solid; yield: 80%.
NMR (300 MHz, δ, D₂O): 5.54-5.37 (m, 1H), 4.96 (d, J=9 Hz, 1H), 4.03-4.36 (m, 4H), 2.68-2.36 (m, 2H), 2.1 (s, 3H), 1.82 (s, 6H), 1.67-1.54 (m, 6H).
MS: 306 [M^{+].}

Preparation of Exemplary Compounds 18 and 19:

The preparation of 1-[2-(adamant-1-ylamino)acetyl]-azetidine-2-carbonitrile (21, EXAMPLE 18), and 1-(2-tert-butylaminoacetyl)azetidine-2-carbonitrile (22, EXAMPLE 19) was conducted according to Scheme 3, below: 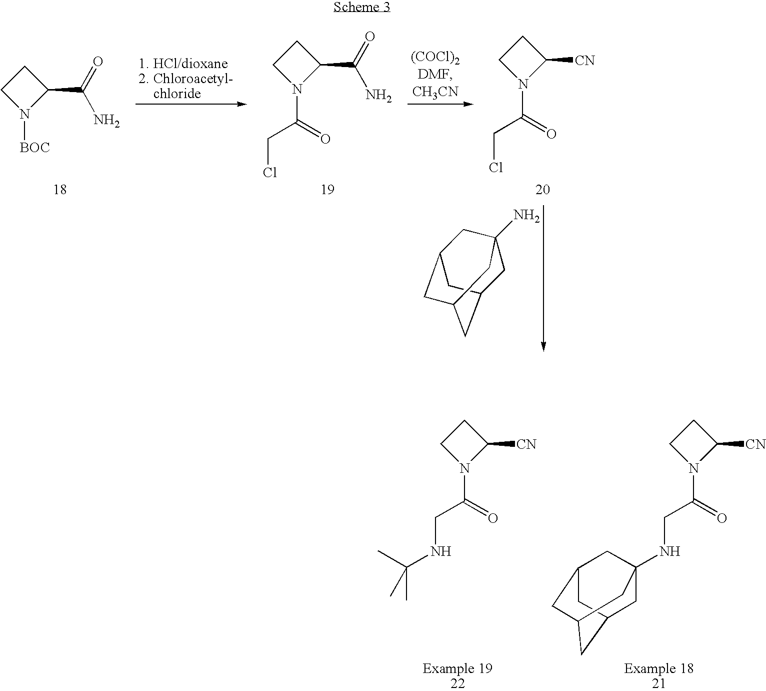
1-(2-Chloro-acetyl)azetidine-2-carboxylic acid amide (19). To a solution of 1-tert-butoxycarbonylazetidine-2-carboxylic acid amide (18, 2.0 g, 10 mmol) in dichloromethane (5 mL) was added HCl solution in dioxane (4M, 20 mL), and the mixture was stirred overnight. The mixture was then concentrated, and the residue taken up in dichloromethane (30 mL) and cooled in an ice bath. The mixture was treated with triethylamine (2.5 g, 25 mmol), followed by dropwise addition of chloroacetyl chloride (1.5 g, 13 mmol). After stirring for 3 h, the mixture was concentrated and the product purified on silica gel eluting with 1:1 hexane:ethyl acetate to obtain 19 as a clear oil; yield 1.5 g (85%).

GCMS: M=176.

TLC: R_{f}=0.2 (1:1 EtOAc:Hexane).

1-(2-Chloro-acetyl)azetidine-2-carbonitrile (20). To a stirred solution of dimethylformamide (0.95 g, 13 mmol) in acetonitrile (50 mL) under argon at 0° C. was added dropwise oxalyl chloride (1.5 g, 12 mmol), and the mixture was stirred for 1 h, followed by dropwise addition of 1-(2-chloro-acetyl)azetidine-2-carboxylic acid amide (1.8 g, 1.0 mmol) solution in acetonitrile (5 mL). After stirring for 15 min, the whole was treated with triethylamine (2.2 g, 22 mmol), and stirring continued for additional 1 h. Solvents evaporated in vacuum, and the product was purified on silica gel eluting with 1:1 hexane:ethyl acetate to obtain 20 as a clear oil; yield 0.5 g (35%).

GCMS: M=158.

TLC: R_{f}=0.3 (1:1 EtOAc:Hexane).

1-[2-(Adamant-1-ylamino)acetyl]-azetidine-2-carbonitrile (21, EXAMPLE 18). Adamant-1-ylamine (1.6 g, 10 mmol) was placed in dimethylformamide (50 mL) and the stirred mixture heated to 70° C. to dissolve the amine. To this was added a solution of 1-(2-chloro-acetyl)-azetidine-2-carbonitrile (0.41 g, 2.6 mmol) in dimethylformamide (5 mL), and the mixture stirred overnight. After evaporation of solvent, product was purified on silica gel eluting with 95:5 CHCl₃:MeOH to obtain 21 as a white solid; yield 0.27 g (38%).

¹H NMR, δ (CDCl₃, 400 MHz): 1.46-1.75 (m, 121); 2.08 (s, 31); 2.61-2.85 (m, 2H); 3.27 (s, 1H); 3.36-3.54 (m, 1H); 3.97-4.39 (m, 2H); 4.77-4.95 (m, 1H).

Anal. Calcd for C₁₆H₂₃N₃O: C, 70.30; H, 8.48; N, 15.37. Found: C, 69.93; H, 8.43; N, 15.23.

TLC: R_{f}=0.3 (95:5 CHCl₃:MeOH).

1-(2-tert-Butylaminoacetyflazetidine-2-carbonitrile (22, EXAMPLE 19) Compound 22 was synthesized by a procedure analogous to that described for Example 18. White solid; yield 0.28 g (56%).

¹H NMR, δ (CDCL3, 400 MHz): 1.11 (s, 9H); 2.61-2.82 (m, 2H); 3.23-3.40 (m, 2H); 4.02-4.35 (m, 2H); 4.80-4.92 (m, 1H).

Anal. Calcd for C₁₀H₁₇N₃O₁: C, 61.51; H, 8.78; N, 21.52. Found: C, 61.31; H, 8.79; N, 21.64.

Preparation of Exemplary Compounds 16, 17, and 20:

The preparation of 1-(octahydroindole-2-carbonyl)azetidine-2-carbonitrile hydrochloride (25, EXAMPLE 20), 1-(2-amino-2-cyclohexyl-acetyl)azetidine-2-carbonitrile (26, EXAMPLE 16), and 1-(pyrrolidine-2-carbonyl)azetidine-2-carbonitrile (27, EXAMPLE 17) was conducted according to Scheme 4, below. 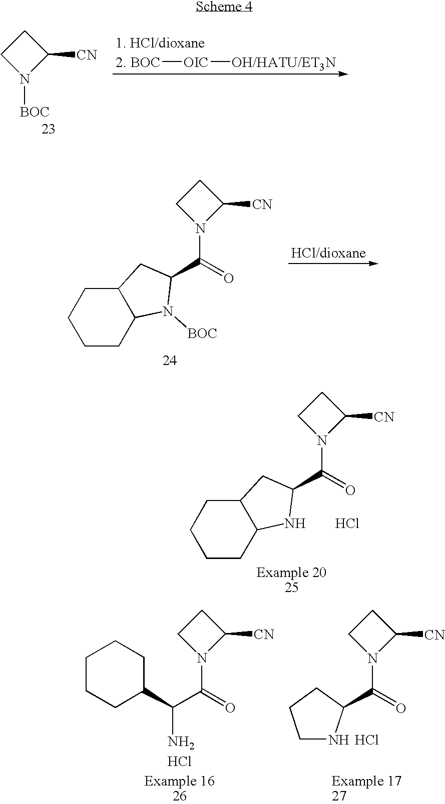
1-(1-tert-butoxycarbonyloctahydroindole-2-carbonyl)azetidine-2-carbonitrile (24). To a solution of 1-tert-butoxycarbonylazetidine-2-carbonitrile (1.0 g, 11 mmol) in dichloromethane (2 mL) was added HCl solution in dioxane (4M, 10 mL), and the mixture stirred overnight. The mixture was then concentrated in vacuum, and the residue was taken up in dimethylformamide (10 mL) and treated with triethylamine (1.7 g, 33 mmol). 1-tert-Butoxycarbonyl-L-octahydroindole-2-carboxylic acid (1.5 g, 11 mmol) and O-(7-azabenzotriazol-1-yl)-N,N,N′,N′-tetramethyluronium hexafluorophosphate (2.7 g, 14 mmol) were added, and the mixture stirred for 3 days. The mixture was then concentrated in vacuum, and the product was purified on silica gel eluting with 1:1 hexane:ethyl acetate to obtain 24 as a clear oil; yield 1.2 g (66%).
¹H NMR, δ (CDCl₃, 400 MHz): 1.04-1.19(m, 1H); 1.24-1.38(m, 2H); 1.43(s, 9H); 1.54-1.79(m, 4H); 1.89-2.21(m, 3H); 2.23-2.36(m, 1H); 2.59-2.80(m, 2H); 3.63-3.90(m, 1H); 3.94-4.41(m, 3H); 4.54-4.94(m, 1H). TLC: R_{f}=0.3 (1:1 EtOAc:Hexane).
1-(octahydroindole-2-carbonyl azetidine-2-carbonitrile hydrochloride (25, EXAMPLE 20). A solution of 1-(1-tert-butoxycarbonyloctahydroindole-2-carbonyl)-azetidine-2-carbonitrile (0.6 g, 1.8 mmol) in 10 mL 4M HCl solution in dioxane (4M, 10 mL) was stirred overnight. The mixture was then concentrated in vacuum, and the residue triturated with ether to give a white solid, which was washed with ether (5×5 mL) and then dried in vacuum to give 25 as a white solid; yield 0.43 g (89%).
¹H NMR, δ (MeOH, 400 MHz): 1.26-1.56 (m, 3H); 1.57-1.82 (m, 4H); 1.84-2.60 (m, 4H); 2.62-2.94 (m, 2H); 3.71-3.86 (m, 1H); 3.99-4.55 (m, 3H); 4.81-4.95 (m, 1H).
Anal. Calcd for C₁₃H₁₉N₃O₁.1.2 HCl.1 H₂O.0.7 Et₂O: C, 53.22; H, 7.80; N, 11.79; Cl, 11.93. Found: C, 53.16; H, 8.01; N, 11.75; Cl, 12.33.
1-(2-Amino-2-cyclohexyl-acetyl azetidine-2-carbonitrile (26, EXAMPLE 16). Compound 26 was synthesized by a procedure analogous to that described for Example 20.
White solid; yield 0.39 g (94%).
¹H NMR, δ (MeOH, 400 MHz): 0.95-1.22 (m, 6H); 1.55-1.83 (m, 5H); 2.55-2.75 (m, 2H); 3.45 (q, J=7.1 Hz, 1H); 3.74-3.82 (m, 1H); 4.20-4.39 (m, 2H); 4.73-4.82 (m, 1H); 4.98-5.06 (m, 1H).
Anal. Calcd for C₁₃H₁₉N₃Oₜ.1 HCl.1H₂O.0.2 Et₂O: C, 52.40; H, 8.11; N, 14.32. Found: C, 52.50; H, 8.11; N, 14.32.
1-(Pyrrolidine-2-carbonyl azetidine-2-carbonitrile (27, EXAMPLE 17). Compound 27 was synthesized by a procedure analogous to that described for Example 20. White solid; yield 0.38 g (51%).
¹H NMR, δ (MeOH, 400 MHz): 1.87-2.05 (m, 3H); 2.08-2.46 (m, 2H); 2.59-2.80 (m, 1H); 3.22-3.40 (m, 2H); 4.13-4.53 (m, 3H); 4.95-5.10 (m, 1H).
Anal. Calcd for C₉H₁₄N₃O₁.1 HCl2.5H₂O.0.4 Et₂O: C, 43.02; H, 7.54; N, 14.20. Found: C, 42.66; H, 7.54; N, 13.83.

Preparation of Exemplary Compound 11:

The preparation of 1-L-α-Cyclohexylglycylthiazolidine hydrogen chloride (29, EXAMPLE 11) was conducted according to the following Scheme 5: 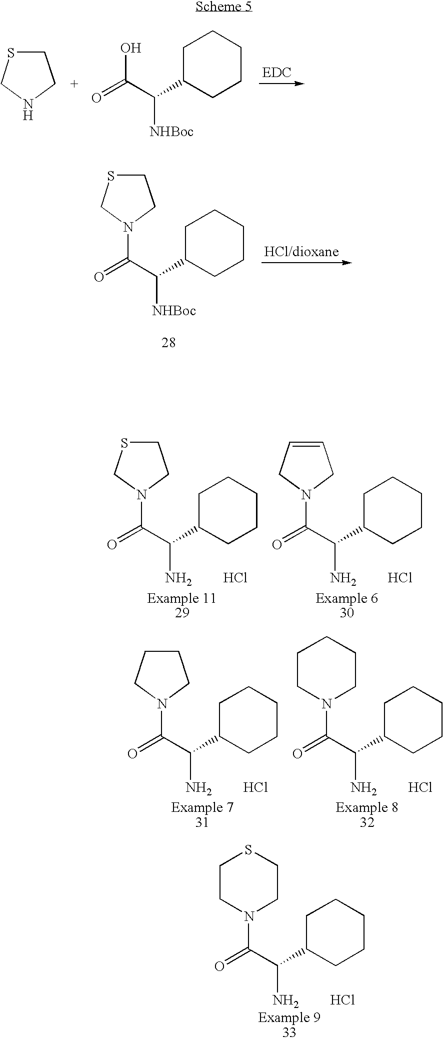
1-(N-BOC-L-α-cyclohexylglycyl)thiazolidine (28). To a stirred solution of thiazolidine (0.11 g, 1.2 mmol) and N-BOC-L-α-cyclohexyl-glycine (0.26 g, 1.0 mmol) in CH₂Cl₂ (20 mL) was added 1-(3-dimethylaminopropyl)-3-ethylcarbodiimide hydrochloride (EDC, 0.23 g, 1.2 mmol) in one portion at room temperature. Stirring was continued overnight at room temperature. The reaction mixture was poured into a mixture of water (35 mL) and ethyl acetate (50 mL). After extraction, the organic layer was separated and washed with water (30 mL) and brine (30 mL), and dried over anhydrous sodium sulfate. Filtration and removal of the solvent in vacuum gave an oil, which was purified by flash column chromatography (silica gel; eluent: ethyl acetate:CH₂Cl₂ 1:4) to give 1-(N-BOC-L-α-cyclohexylglycyl)thiazolidine (28) (0.20 g, yield: 61%).
MS: 327 [M+1].
1-(L-α-Cyclohexylglycyl)thiazolidine hydrogen chloride (29, EXAMPLE 11). 1-(N-BOC-L-α-cyclohexylglycyl)thiazolidine (28, 0.20 g, 0.61 mmol) was added in one portion to a stirred solution of HCl in diethyl ether (2M, 10 mL) under nitrogen. The reaction mixture was stirred for 4 h at room temperature. After completion of the reaction, the liquid phase was decanted, and the solid was washed with dry diethyl ether (3×10 mL), filtered, and then dried in vacuum to give 29 as a yellow solid; yield 0.14 g (87%).
¹H NMR, δ (300 MHz, D₂O): 4.30-4.64 (m, 2H), 3.55-4.06 (m, 3H), 2.96-3.06 (m, 2H), 1.50-1.80 (m, 6H), 1.00-1.20 (m, 5H).
Anal. Calcd. for C₁₁H₂₀N₂OS.1.35 ClH.(0.5H₂O): C, 45.22; H, 7.71; N, 9.59; S, 10.97; Cl, 16.38. Found: C, 45.38; H, 7.69; N, 9.45; S, 10.48; Cl, 16.19.
MS: 229 [M+1].
1-(L-α-cyclohexylglycyl)-3-pyrroline hydrogen chloride (30, EXAMPLE 6). The procedure used for the preparation of the title compound is analogous to that used for compound 29, except that during the coupling reaction in the first step, 3-pyrroline was used instead of thiazolidine to provide the required moiety on the upper part of a molecule.
White solid; yield 57%.
¹H NMR (300 MHz, δ, D₂O): 5.80-5.86 (m, 21), 4.30-4.40 (m, 2H), 4.10-4.28 (m, 2H), 3.99 (d, J=6.7 Hz, 1H), 1.80-1.95 (m, 1H), 1.52-1.72 (m, 5H), 1.00-1.26 (m, 5H)
Anal. Calcd. for C₁₂H₂₀N₂O.1 ClH(0.2H₂O): C, 58.03; H, 8.68; N, 11.28; Cl, 14.27. Found: C, 58.35; H, 8.59; N, 11.22; Cl, 14.01.
MS: 209 [M+1].
1-(L-α-cyclohexylglycyl)pyrrolidine hydrogen chloride (31, EXAMPLE 7). The procedure used for the preparation of the title compound is analogous to that used for compound 29, except that during the coupling reaction in the first step pyrrolidine was used instead of thiazolidine to provide required moiety on the upper part of a molecule.
White solid; yield 58%.
¹H NMR (300 MHz, δ, D₂O): 4.15-4.25 (m, 1H), 3.65-3.75 (m, 2H), 3.45-3.65 (m, 2H), 2.00-2.08 (m, 51), 1.78-1.90 (m, 5H), 1.30-1.34 (m, 5H)
Anal. Calcd. for C₁₂HnN₂O.1ClH: C, 58.4; H, 9.39; N, 11.35; Cl, 14.37. Found: C, 58.58; H, 9.61; N, 11.27; Cl, 14.09.
MS: 211 [M+l].
1-(L-α-cyclohexylglycyl)piperidine hydrogen chloride (32, EXAMPLE 8). The procedure used for the preparation of the title compound is analogous to that used for 29, except that during the coupling reaction in the first reaction step, piperidine was used instead of thiazolidine to provide the required moiety on the upper part of a molecule.
White solid; yield 58%.
¹H NMR (300 MHz, δ, D₂O): 4.20-4.30 (m, 1H), 3.25-3.55 (m, 4H), 1.30-1.70 (m, 10H), 0.90-1.25 (m, 7H)
Anal. Calcd. for C₁₃H₂₄N₂O.1.8ClH.(0.6H₂O): C, 49.79; H, 8.68; N, 8.93; Cl, 20.35.
Found: C, 49.29; H, 8.14; N, 8.68; Cl, 20.26.
MS: 225 [M+l].
(1-L-α-Cyclohexylglycyl)thiomorpholine hydrogen chloride (33, EXAMPLE 9). The procedure used for the preparation of the title compound is analogous to that used for 29, except that during the coupling reaction in the first reaction step, thiomorpholine was used instead of thiazolidine to provide the required moiety on the upper part of a molecule.
Yellow solid; yield 31%.
¹H NMR (300 MHz, δ, D₂O): 4.30 (d, J=3.5 Hz, 1H), 3.82-3.73 (m, 4H), 2.72-2.60 (m, 4H), 1.95-1.55 (m, 6H), 1.35-1.05 (m, 51)
Anal. Calcd. for C₁₂HnN₂OS.1C1H.(0.4H₂O): C, 50.39; H, 8.39; N, 9.79; S, 11.21; Cl, 12.39. Found: C, 50.2; H, 8.3; N, 9.62; S, 11.16; Cl, 12.36.
MS: 243 [M+1].

Preparation of Exemplary Compound 4:

The preparation of 2-(L)-Cyano-(octahydro-isoindolyl)pyrrolidine hydrogen chloride (35, EXAMPLE 4) was conducted according to Scheme 5a, below.

(R,S)—N-BOC-octahydroisoindole-1-carboxylic acid (33). A mixture of (R,S)—N-BOC-1,3-dihydro-2H-isoindole-1-carboxylic acid (1.0 g, 3.8 mmol), PtO₂ (0.1 g) and acetic acid (25 mL) was hydrogenated at 50 psi at room temperature for 3 h. The reaction mixture was filtered through Celite plug, and solvents removed in vacuum to give 33 as off-white solid; yield 1.0 g (98%).
MS: 270 [M+1]. 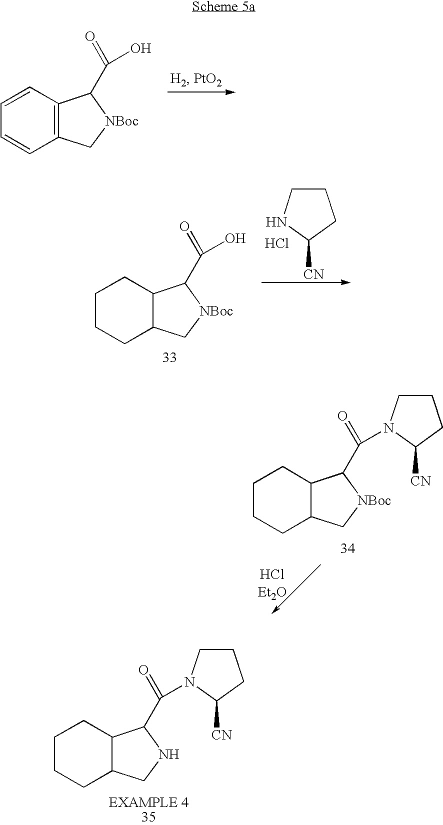
(R,S)—N-BOC-(octahydroisoindolyl)-2-(Q-cyano-pyrrolidine (34). To a stirred mixture of 2-(L)-cyanopyrrolidine hydrogen chloride (93 mg, 0.70 mmol), diisopropylethylamine (0.49 mL, 2.8 mmol) and 33 (135 mg, 0.50 mmol) in dry DMF (5 mL) was added O-(7-azabenzotriazol-1-yl)-N,N,N′,N′-tetramethyluronium hexafluorophosphate (HATU, 266 mg, 0.70 mmol) in one portion at room temperature. The resulting mixture was stirred overnight at room temperature. Solvent was removed in vacuum; water (30 mL) and ethyl acetate (50 mL) were added to the −67mixture. After extraction, the organic layer was separated, washed with water (30 mL) and brine (30 mL), and dried over anhydrous sodium sulfate. Removal of the solvent in vacuum gave an oil, which was purified by flash column chromatography (silica gel; eluent: ethyl acetate:CH₂Cl₂ 1:4) to give 34; yield 0.1 g (59%).
MS: 348 [M+1].
2-(L)-Cyano-(octahydro-isoindolyl)pyrrolidine hydrogen chloride (35, EXAMPLE 4). The procedure used for deprotection of 34 to prepare the title compound was analogous to that used for 29. White solid; yield 0.071 g (86%).
¹H NMR (300 MHz, δ, D₂O): 4.35-4.40 (m, 1H), 4.10-4.25 (m, 1H), 3.40-3.50 (m, 1H), 3.20-3.40 (m, 1H), 3.10-3.20 (m, 2H), 2.30-2.55 (m, 2H), 1.74-2.06 (m, 4H), 1.22-1.48 (m, 4H), 0.80-1.02 (m, 4H)
Anal. Calcd. for C₁₄H₂₁N₃O.1.55ClH.(2.6H₂O): C, 47.95; H, 7.98; N, 11.98; Cl, 15.78. Found: C, 47.81; H, 7.53; N, 12.19; Cl, 15.67.
MS: 248 [M+1].

Preparation of Exemplary Compound 12:

The preparation of 1-[2-(adamant-1-ylamino)acetyl]-3-thiopyrrolidine (37, EXAMPLE 12) was conducted according to Scheme 6, below. 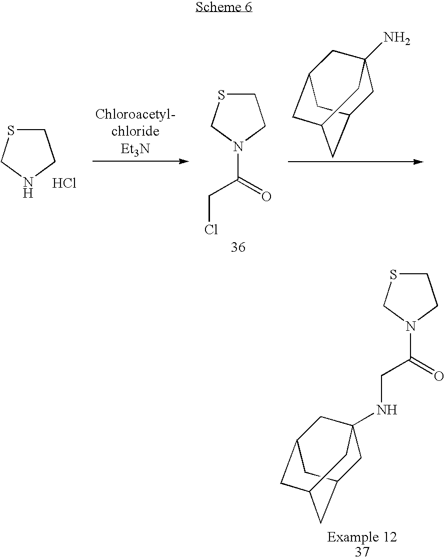
1-(2-Chloro-acetyl)-3-thiopyrrolidine (36). To a suspension of 3-thiopyrrolidine hydrochloride (1.26 g, 10 mmol) in dichloromethane (5 mL) was added triethylamine (2.51 g, 25 mmol), followed by dropwise addition of chloroacetyl chloride (1.52 g, 13 mmol). After stirring for 3 h, the mixture was concentrated and the product purified on silica gel eluting with 1:1 hexane:ethyl acetate to obtain 36 as a yellow oil; yield 1.22 g (73%). GCMS: M=166.
1-[2-(adamant-1-ylamino)acetyl]-3-thiopyrrolidine (37, EXAMPLE 12). Adamant-1-ylamine (2.4 g, 15 mmol) was placed in dimethylformamide (75 mL) and the stirred mixture heated to 70° C. to dissolve the amine. To this was added a solution of 1-(2-chloro-acetyl)-3-thiopyrrolidine (36, 0.83 g, 5 mmol) in dimethylformamide (15 mL), and the mixture stirred overnight. After evaporation of solvent, product was purified on silica gel eluting with 95:5 EtOAc:MeOH to obtain 37 as a yellow wax; yield 0.62 g (44%).
¹H NMR, δ (CDCl₃, 400 MHz): 4.6 (s, 2H); 3.86 (t, J=5.2 Hz, 2H); 3.70 (s, 2H); 3.45 (t, J=5.0 Hz, 2H); 2.08 (br.s, 5H); 1.59 (br. S, 10H).
Anal. Calcd for C₁₅H₂₄N₂OS: C, 64.24; H, 8.63; N, 9.99. Found: C, 63.90; H, 8.71; N, 10.11.

Preparation of Exemplary Compounds 1, 2, 5, and 10:

The preparation of 1-(octahydroindole-2-carbonyl)pyrrolidine-2-carbonitrile hydrochloride (39, EXAMPLE 1), 1-(3-azabicyclo[3.1.0]hexane-2-carbonyl)pyrrolidine-2-carbonitrile hydrochloride (40, EXAMPLE 2), 3-(octahydroindole-2-carbonyl)thiazolidine4-carbonitrile hydrochloride (41, EXAMPLE 5), and 1-(octahydroindole-2-carbonyl)pyrrolidine hydrochloride (42, EXAMPLE 10) was conducted according to Scheme 7, below.

1-01-BOC-octahydroindole-2-carbonyl)pyrrolidine-2-carbonitrile (38). To a solution of N-BOC-L-octahydroindole-2-carboxylic acid (1.0 g, 3.7 mmol) in acetonitrile (50 mL) was added diisopropylethylamine (1.1 g, 8.3 mmol), O-(7-azabenzotriazol-1-yl)-N,N,N′,N′-tetramethyluronium hexafluorophosphate (HATU, 1.68 g, 4.2 mmol), followed by addition of cyanopyrrolidine hydrochloride (0.5 g, 3.7 mmol). The −69mixture was stirred overnight, solvent stripped off, and the crude material purified by flash chromatography (silica gel, eluent: EtOAc:hexanes 2:1). Clear oil; yield 0.78 g (60%).

MS: 348 [M+1]. 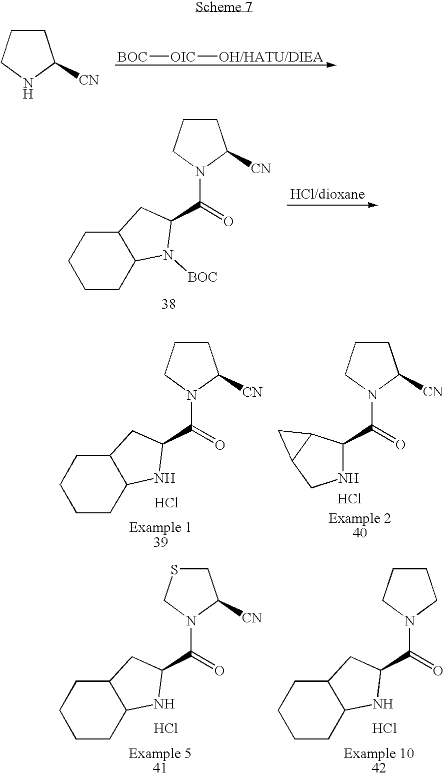
1-(octahydroindole-2-carbonyl pyrrolidine-2-carbonitrile hydrochloride (39, EXAMPLE 1). Compound 38 (0.78 g, 2.2 mmol) was stirred overnight in HCl solution in dioxane (4M, 50 mL) at room temperature. Product was precipitated out with ethyl ether, solvents decanted, and the precipitate washed twice with ethyl ether. After drying under vacuum, off-white solid was obtained. Yield 0.45 g (73%).
¹H NMR (300 MHz, δ, MeOD): 4.86-4.82 (m, 1H), 4.61 (t, J=6 Hz, 1H), 3.80-3.75 (m, 1H), 3.64 (t, J=6 Hz, 2H), 2.62-2.48 (m, 2H), 2.36-2.20 (m, 4H), 2.14-1.85 (m, 2H), 1.77-1.68 (m, 4 H), 1.50-1.41 (m, 3H)
MS: 283 [M+].
1-(3-azabicyclo[3.1.0]hexane-2-carbonyl)pyrrolidine-2-carbonitrile hydrochloride (40, EXAMPLE 2). The procedure used for its preparation is analogous to that used for Example 1, except 1-(3-azabicyclo[3.1.0]hexane-2-carboxylic acid was used instead of BOC-OIC-OH acid to provide the required moiety in the lower part of the molecule. Yellowish wax; yield 0.51 g (68%).
¹H NMR (300 MHz, δ, MeOD): 1.43-1.46 (m, 3H); 1.63-1.75 (m, 4H); 1.91-2.05 (m, 6H); 2.44-2.60 (m, 2H); 3.41-3.66 (m, 5H); 3.72-3.77 (m, 1H); 4.52-4.58 (m, 1H).
MS: 241 [M+].
3-(octahydroindole-2-carbonyl)thiazolidine-4-carbonitrile hydrochloride (41, EXAMPLE 5). The procedure used for its preparation is analogous to that used for Example 1, except 4-cyano-1-thiazolidine was used instead of 2-cyanopyrrolidine to provide the required moiety in the upper part of the molecule. Yellow solid; yield 0.20 g (77%).
¹H NMR (300 MHz, δ, MeOD): 1.35-1.50 (m, 41); 1.65-1.78 (m, 4H); 1.93-2.09 (m, 2H); 2.53-2.65 (m, 2H); 3.41-3.44 (m, 2H); 3.66-3.83 (m, 2H) 4.69-4.82 (m, 3H); 5.84-5.87 (m, 1H).
MS: 301 [M+].
1-(octahydroindole-2-carbonyl)pyrrolidine hydrochloride (42, EXAMPLE 10). The procedure used for its preparation is analogous to that used for Example 1, except pyrrolidine was used instead of 2-cyanopyrrolidine to provide the required moiety in the upper part of the molecule. White solid; yield 0.38 g (70%).
¹H NMR (300 MHz, δ, MeOD): 0.68-0.77 (m 1H); 0.94-1.05 (m, 1H); 1.86-2.86 (m, 6H); 3.42-3.50 (n, 1H1); 3.42-3.50 (m, 1H); 3.66-3.83 (m, 4H); 4.624.66 (m, 1H).
MS: 258 [M+].

Measuring the Bioactivity of the Compounds of the Invention:

As noted above, a number of methods can be used to assay for the bioactivity of the compounds of the invention. Appropriate assays can be in vivo or in vitro methods, which are themselves well-established in the art. The literature cited above discloses many such assays, and can be relied upon to make and practice aspects of the instant invention. The examples below illustrate assays for the ability of the compounds to protect neuronal cells from toxic treatments and the ability of the compounds to elicit neuronal cell growth, regeneration, or neurite extension.

Measuring DPP IV Inhibition:

Dilute 1 volume of rat plasma (coagulated with sodium citrate) to 2.5 volumes assay buffer (25 mM HEPES, 140 mM NaCl, 1% BSA [added on the day of the assay]; pH 7.8) to yield approximately 350 micrograms total protein/well in a 96-well plate. Prepare 80 mM MgCl₂ solution in assay buffer (16.264 mg/mL). Dilute the peptide substrate (H-Gly-Pro-alpha methyl coumarin, 10 mM stock in 100% DMSO) 1:100 in assay buffer. Compounds of this invention are diluted in 100% DMSO. Add 10 microliter of diluted compounds or DMSO vehicle to wells. Add 25 uL of rat plasma or buffer to wells. Add 25 uL MgCl₂ to all wells. Vortex gently for 1 minute, then pre-incubate at room temp for 10 minutes. Start the reaction by adding 50 uL peptide substrate (no vortexing), and incubate the plate at room temp in the dark for 30 minutes. Stop the reaction by adding 25 uL of 25% glacial acetic acid. Read the plate at 380 nm (excitation) and 460 nm (emission). Plot absorbance vs. concentration of test compound to determine the concentration of test compound which yields a 50% inhibition of DPP IV enzymatic activity (IC50).

The following Table 1 gives such IC50 values, as determined for exemplary compounds of this invention.

TABLE 1

| Compound | IC50 (nM) |
|---|---|
| Example 1 | 13.9 |
| Example 2 | 60.7 |
| Example 3 | 1.65 |
| Example 4 | 551 |
| Example 5 | 3.8 |
| Example 6 | 1,880 |
| Example 7 | 125 |
| Example 8 | 22,200 |
| Example 9 | 10,300 |
| Example 10 | 10,000 |
| Example 11 | n.d. |
| Example 12 | 4,820 |
| Example 13 | 6.2 |
| Example 14 | 6.5 |
| Example 15 | 62.1 |
| Example 16 | 1,200 |
| Example 17 | 1,100 |
| Example 18 | 223 |
| Example 19 | 337 |
| Example 20 | 178 |

Neuroprotection Assay in Spinal Cord Slice Preparations: All cultures are derived from postnatal day 8 (P8) Sprague-Dawley rat lumbar spinal cord slices of 325 micron thickness, prepared using a commercially available Mcllwain tissue chopper. Experiments consist of two 6-well plates with 5 slices from 4 different animals per well; slices are cultured at the media/atmosphere interface on a commercially available permeable membrane culture well insert. Media changes are performed every 3 to 4 days. Cultures are treated with the neurotoxin THA [L(−)-threo-3-hydroxyaspartic acid; Tocris Cookson Inc., Ballwin, Mo.] at 200 μM+compound (10) after one week in culture. The control is an untreated sample with 0.1% DMSO as vehicle. The THA control is a THA treated sample with 0.1% DMSO as vehicle. Two wells are used per condition. One media change with new THA and compounds is performed. The experiment is stopped 6 to 8 days following drug treatment (13-15 total days in vitro, DIV) as dictated by visual assessment of lesion, by fixation with 4% paraformaldehyde/0.1 M phosphate buffer for 30 minutes. Slices are permeabilized with 100% cold methanol for 10 minutes and transferred to staining wells. The slices are blocked with 10% HS/TBS (horse serum/tris-buffered saline). Primary antibody incubation is overnight at 4° C. with SMI-32 antibody 1:5000 in 2% HS/TBS. SMI-32 is specific towards the unphosphorylated H neurofilament subunit. Vectastain ABC Elite Kit with rat absorbed anti-mouse secondary antibody is used with 3,3-diaminobenzidine as a chromogen to stain the slices. The slices are mounted onto a slide and a coverslip is sealed with DPX mounting solution.

Quantification of surviving neurons is performed on a Zeiss Axiovert microscope. Neuronal survival is determined by observing an intact neuronal cell body with processes located ventrally of the central canal in each hemisphere. This correlates to laminae VII, VIII and IX. Each hemisphere is counted individually. Statistical analysis is performed with StatView™ software on a minimum of three different experiments per condition and significance is determined as compared to THA control. The percent of protection is determined from the average number of living neurons by the following equation: (drug treatment condition—THA control)/(Untreated control-THA control).

THA-treated control cultures display a significantly reduced average number of SMI-32 immunoreactive neurons per ventral hemisphere of the spinal cord slices at the end of the culturing interval, as compared to untreated control cultures. Addition of the compounds of this invention to THA-treated cultures causes a significant protection from THA-induced cell death.

In Vivo Reinnervation of the Denervated Striatum by Nigrostriatal Dopaminergic Fibers: The MPTP-lesioned mouse model of Parkinson's disease was utilized to demonstrate in vivo efficacy of the compounds of this invention. MPTP (N-methyl-4-phenyl-1,2,3,6-tetrahydropyridine) is a systemically available neurotoxin specific to nigrostriatal dopaminergic neurons, i.e. to the cells that degenerate in human Parkinson's disease. Administration of MPTP to mice leads to a selective partial destruction of the mesotelencephalic dopaminergic projection, and to a loss of dopamine and dopaminergic fibres in the corpus striatum, which is the main forebrain target of midbrain dopaninergic neurons.

Young adult male CD1 albino mice (Harlan-Sprague Dawley; 22-25 g) were dosed i.p. with the dopamine cell-specific neurotoxin N-methyl-4-phenyl-1,2,3,6-tetrahydropyridine (MPTP hydrochloride, calculated as 34 mg/kg free base), dissolved in saline at a concentration of 3.4 mg/ml free base once daily on days one to five.

Experimental compounds were administered once daily on days 1-5 (10 mg/kg in Intralipid vehicle, s.c.), one hour prior to MPTP-administration. On day seven, animals were perfused transcardially with 10% neutral buffered formalin. Sagittal sections of striatal tissue were cut at 20 μm thickness on a freezing microtome and processed for free-floating tyrosine hydroxylase immunocytochemistry using a polyclonal TH antibody (Pel Freeze, 1:2500 under refrigeration for 4 nights), further processed using the avidin:biotin peroxidase method (Vector Elite kit), and visualized with Diamino benzidine (DAB-HCl, Polysciences).

Blinded analysis of TH fiber density in the central striatum was performed at 630× magnification. For each mouse striatum, five representative 100 μm×100 μm fields in the central striatum were photographed using a digital video camera. The percentage of sample field covered by TH positive processes and terminals was calculated using an image analysis program (“Simple,” Compix Inc., Pittsburgh, Pa.). The mean striatal innervation density was calculated for each group. The magnitude of striatal deafferentation due to the MPTP lesion was assessed by dividing the observed striatal innervation values obtained in MPTP/vehicle treated cases by the mean striatal innervation density in the Vehicle/Vehicle group and expressed as % loss. The relative efficacy of the compounds of this invention was expressed as % protection of striatal innervation density, i.e., the degree to which the density of TH positive fibres in the striatum of lesioned/compound-treated animals exceeded the loss observed in lesioned-alone animals.

Experimental animals treated with Exemplary Compound 1 of this invention according to the above protocol displayed a 32.5% protection of striatal tyrosine hydroxylase-immunorecative fibres. Treatment with Exemplary Compound 20 resulted in a 32.6% protection of striatal tyrosine hydroxylase-immunoreactive fibres relative to control animals. Administration of other compounds of this invention is expected to lead to a significant protection of striatal dopaminergic innervation density from neurotoxin-induced lesion.

The specific examples disclosed herein should not be interpreted as a limitation to the scope of the invention. Instead, they are merely exemplary embodiments one skilled in the art would understand from the entire disclosure of this invention. The invention being thus described, it will be obvious that the same may be varied in many ways. Such variations are not to be regarded as a departure from the spirit and scope of the invention and all such modifications are included to be within the scope of the following claims.

1. A compound of Formula I: 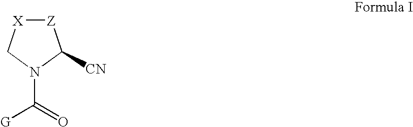
and pharmaceutically acceptable derivatives thereof,
wherein the pyrrolidine ring formed by X, Z, N, and the carbon atoms to which they are attached, is saturated, or optionally contains one double bond;
X is selected from the group consisting of CH₂, CH, S, O, NH, N, C═O, CF₂, CF, CH—Y, and C—Y;
Z is selected from the group consisting of CH₂, CH, CF₂, CF, C—Y and CH—Y;
wherein Y is halogen, hydroxy, or C₁-C₃ alkyloxy; and
wherein one of X or Z must be CH₂; or CH if said pyrrolidine ring contains one double bond;
and where G is 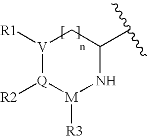
wherein M, Q, and V represent carbon atoms;
n is 0 or 1; and where either
R1 and R2, taken together with V and Q, or
R2 and R3, taken together with Q and M, form a 3-6 membered, saturated carbocyclic or heterocyclic ring which may contain one or two heteroatoms selected from the group consisting of O, S, and N.

2. The compound of claim 1, wherein said pyrrolidine ring is saturated; n is 0; Y is fluoro or C₁-C₃ alkyloxy; and said 3-6 membered saturated ring is a carbocyclic ring.

3. A compound of Formula II: 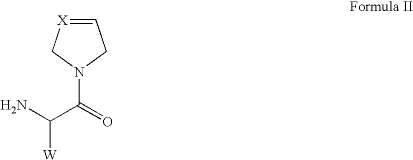
and pharmaceutically acceptable derivatives thereof,
where X is as defined for Formula I above, and X may further be:
—S—CH₂—, —S—CH══, —CH₂—S—, (CH₂)₂, and —CH₂—CH══, and
where W is either W′ or W″;
wherein W′ is a saturated cyclic hydrocarbon; and
W″ is a non-cyclic straight or branched chain alkyl group,
and the dashed bond symbol represents an optional bond;
provided that: when X is S, then W″ is not

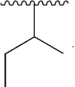

4. A compound of Formula IIa: 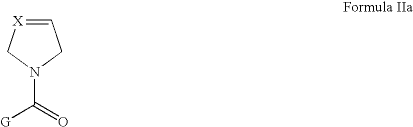
and pharmaceutically acceptable derivatives thereof,
where the dashed bond symbol represents an optional bond,
X is defined as for Formula II above;
the substituent G is defined as for Formula I above;
n in said substituent G is 0; and
the 3-6 membered saturated ring in said substituent G is a carbocyclic ring.

5. A compound of Formula III: 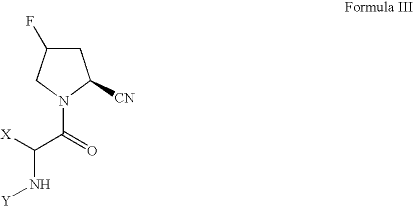
and pharmaceutically acceptable derivatives thereof;
where X and Y may independently be H, or W as defined for Formula II of claim 3 above; provided that:
when Y is H, then X is W; and
when X is H, then Y is W; and
X and Y may not both be W.

6. A compound of Formula IVa: 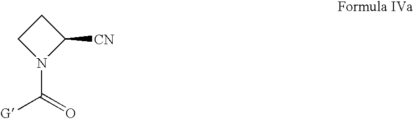
and pharmaceutically acceptable derivatives thereof;
where G′ is a group G as defined for Formula I of claim 1 above; or where G′ is a group:

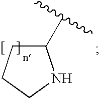

7. A compound of Formula IVb: 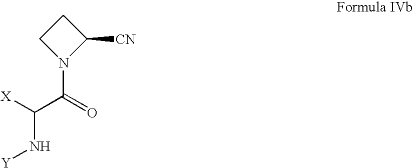
and pharmaceutically acceptable derivatives thereof;
where X and Y may independently be H, or W as defined for Formula II of claim 3 above; provided that:
when Y is H, then X is W; and
when X is H, then Y is W; and
X and Y may not both be W.

8. A pharmaceutical composition, comprising a compound of Formula I according to claim 1, and a pharmaceutically acceptable carrier, diluent, or excipient.

9. A pharmaceutical composition, comprising a compound of Formula I according to claim 2, and a pharmaceutically acceptable carrier, diluent, or excipient.

10. A pharmaceutical composition, comprising a compound of Formula II according to claim 3, and a pharmaceutically acceptable carrier, diluent, or excipient.

11. A pharmaceutical composition, comprising a compound of Formula IIa according to claim 4, and a pharmaceutically acceptable carrier, diluent, or excipient.

12. A pharmaceutical composition, comprising a compound of Formula III according to claim 5, and a pharmaceutically acceptable carrier, diluent, or excipient

13. A pharmaceutical composition, comprising a compound of Formula IVa according to claim 6, and a pharmaceutically acceptable carrier, diluent, or excipient.

14. A pharmaceutical composition, comprising a compound of Formula IVb according to claim 7, and a pharmaceutically acceptable carrier, diluent, or excipient.

15. A pharmaceutical composition, comprising a compound of Formula I, II, IIa, III, IVa, or IVb, and a pharmaceutically acceptable carrier, diluent, or excipient, wherein the composition is further comprising an additional agent selected from antidiabetic agents, contraceptive agents, anti-inflammatory agents, immunosuppressive agents, anti-AIDS agents, anti-osteoporosis agents, anticancer agents, and anti-obesity agents.

16. A method for treating a medical condition, comprising: administering to a patient in need of such treatment a therapeutically effective amount of a compound of Formula I according to claim 1.

17. A method for treating a medical condition, comprising: administering to a patient in need of such treatment a therapeutically effective amount of a compound of Formula I according to claim 2.

18. A method for treating a medical condition, comprising: administering to a patient in need of such treatment a therapeutically effective amount of a compound of Formula II according to claim 3.

19. A method for treating a medical condition, comprising: administering to a patient in need of such treatment a therapeutically effective amount of a compound of Formula IIa according to claim 4.

20. A method for treating a medical condition, comprising: administering to a patient in need of such treatment a therapeutically effective amount of a compound of Formula III according to claim 5.

21. A method for treating a medical condition, comprising: administering to a patient in need of such treatment a therapeutically effective amount of a compound of Formula IVa according to claim 6.

22. A method for treating a medical condition, comprising: administering to a patient in need of such treatment a therapeutically effective amount of a compound of Formula IVb according to claim 7.

23. A method for treating a medical condition, comprising: administering to a patient in need of such treatment a therapeutically effective amount of a compound of Formula I, II, IIa, III, IVa, or IVb, wherein said medical condition is a neurological disorder, diabetes, insulin resistance, hyperglycemia, hyperinsulinemia, elevated blood levels of free fatty acids or glycerol, obesity, hypertriglyceridemia, atherosclerosis, impaired glucose tolerance, impaired glucose homeostasis, polycystic ovary syndrome, arthritis, allograft rejection in organ or tissue transplantation, autoimmune disorder, AIDS, inflammatory bowel disease, osteoporosis, psoriasis, metastatic cancer, or rheumatoid arthritis.

24. The method of claim 23, wherein said neurological disorder is a neurodegenerative disorder; neuropathic disorder; neurovascular disorder;
traumatic injury of the brain, spinal cord, or peripheral nervous system;
demyelinating disease of the central or peripheral nervous system; metabolic or hereditary metabolic disorder of the central or peripheral nervous system; or
toxin-induced- or nutritionally related disorder of the central or peripheral nervous system.

25. The method of claim 24, wherein said neurodegenerative disorder is Parkinson's disease, Alzheimer's disease, amyotrophic lateral sclerosis (ALS), Huntington's disease, cerebellar ataxia, or multisystem atrophy.

26. A method for treating a neurological disorder, comprising:
administering to a patient in need of such treatment a therapeutically effective amount of a compound of Formula II: 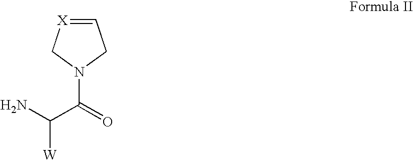
and pharmaceutically acceptable derivatives thereof,
where X is as defined for Formula I above, and X may further be:
—S—CH₂—, —S—CH══, —CH₂—S—, (CH₂)₂, and —CH₂—CH══, and where
W is either W′ or W″;
wherein W′ is a saturated cyclic hydrocarbon; and
W″ is a non-cyclic straight or branched chain alkyl group,
and the dashed bond symbol represents an optional bond.

27. The method according to claim 26, wherein said neurological disorder is a neurodegenerative disorder; neuropathic disorder; neurovascular disorder;
traumatic injury of the brain, spinal cord, or peripheral nervous system;
demyelinating disease of the central or peripheral nervous system; metabolic or hereditary metabolic disorder of the central or peripheral nervous system; or
toxin-induced- or nutritionally related disorder of the central or peripheral nervous system.

28. The method according to claim 27, wherein said neurodegenerative disorder is Parkinson's disease, Alzheimer's disease, amyotrophic lateral sclerosis (ALS), Huntington's disease, cerebellar ataxia, or multisystem atrophy.

29. A method for treating a neurological disorder, comprising:
administering to a patient in need of such treatment a therapeutically effective amount of a compound of Formula V: 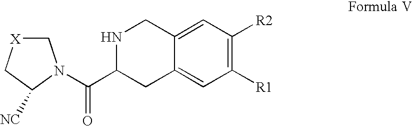
or of a pharmaceutically acceptable derivative thereof;
wherein X is CH₂, S, O, and C(CH₃)₂;
and R1 and R2 are independently selected from the group consisting of hydrogen, hydroxy, C₁-C₈ straight or branched chain alkyl, alkyl, alkoxy, aralkoxy, and halogen.

30. A method for treating a neurological disorder, comprising:
administering to a patient in need of such treatment a therapeutically effective amount of a compound of Formula VI: 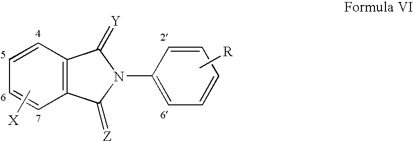
or of a pharmaceutically acceptable derivative thereof;
wherein the dashed bond symbol represents an optional bond;
X, if present, is a single substituent at one, or multiple substituents at several of positions 4-7; and is independently selected from the group consisting of nitro, amino, hydroxy, and halo;
Y and Z are independently O or S;
R is a single substituent at position 2′ or 6′, or two substituents at positions 2′ and 6′, and is independently selected from the group consisting of C₁-C₄ straight or branched chain alkyl, C₁-C₄ straight or branched alkoxy, C₁-C₄ straight or branched alkylthio, aminomethyl, and aminoethyl.

31. A method for treating a neurological disorder, comprising:
administering to a patient in need of such treatment a therapeutically effective amount of a compound of Formula VII: 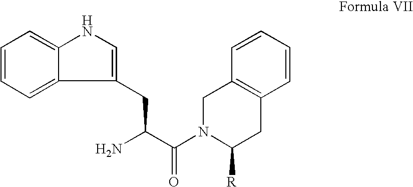
or of a pharmaceutically acceptable derivative thereof;
wherein R is a carboxy group, or an amino acid selected from the group consisting of Ala, Arg, Asp, Asn, Glu, Gln, Gly, His, Ile, Leu, Lys, Met, Phe, Pro, Ser, Thr, Trp, Tyr, Val, and Cys.

32. A method of treating a neurological disorder, comprising: administering to a patient in need of such treatment a therapeutically effective amount of a compound of Formula VIII: 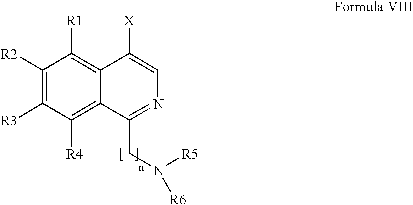
or of a pharmaceutically acceptable derivative thereof;
wherein n is 1 or 2;
R1, R2, R3, and R4 are independently hydrogen, methoxy, ethoxy, or propoxy;
R5 and R6 are independently hydrogen or methyl; and
X is —(CO)—OEt; —CH═CH—(CO)—OEt; —CH₂—CH₂—(CO)—OEt; —COOH;
—CONH₂; —CONH-Prop; —NH—(CO)—OEt; —CH₂—OH; CHO; or
—CH₂—(CO)—OEt.

33. A method of treating a neurological disorder, comprising: administering to a patient in need of such treatment a therapeutically effective amount of a 2-cyanopyrrolidine compound of Formula IX: 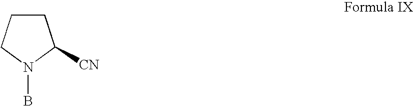
or of a pharmaceutically acceptable derivative thereof;
wherein one or two of the bonds in the 2-cyanopyrrolidine ring is a double bond; and
B is any alpha or beta amino acid connected to the ring with an amide or peptide bond.

34. The method of claim 33, wherein B in said compound of Formula IX is B′ or B″: 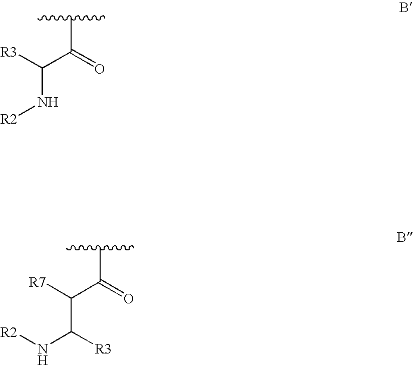
wherein R2, R3, and R7 are independently C₁-C₁₀ alkyl, C₂-C₁₀ alkenyl, C₂-C₁₀ alkynyl, C₃-C₁₀ cycloalkyl, C₅-C₁₀ cycloalkenyl, aryl, heteroaryl, or hydrogen; provided, however, that R2 and R3 in B′ may not both be hydrogen; and that R2, R3, and R7 in B″ may not all be hydrogen;
where R7 in B″ may further be halogen, C₁-C₁₀ alkoxy, C₁-C₁₀ alkylthio, C₁-C₁₀ alkylamino, C₁-C₁₀ dialkylamino, hydroxymethyl, nitro, trifluoromethyl, trifluoromethoxy, trifluoromethylthio, N-hydroxyimino, cyano, carboxy, acetamido, hydroxy, sulfamoyl, or carbamoyl;
wherein said alkyl, alkenyl, alkynyl, cycloalkyl, or cycloalkenyl, are optionally and independently substituted with one or more R4; and wherein said aryl or heteroaryl are optionally and independently substituted with one or more R5; and wherein said aryl or heteroaryl in R3 is optionally fused to a C₃-C₁₀ cycloalkane;
R2 is optionally connected to R3, or R7 if present, by a single bond, or by a saturated or unsaturated bridge containing 1-3 atoms selected from the group consisting of carbon, nitrogen, oxygen, and sulphur; thus forming a ring, which is optionally fused to an aryl or heteroaryl, said aryl or heteroaryl being optionally substituted with one or several R5 independently;
R4, if present, is cycloalkyl, aryl optionally substituted with one or more R5 independently, heteroaryl optionally substituted with one or more R5 independently, amino optionally substituted with one or more R6 independently, —SO—R6, —SO₂—R6, —CO—R6, —COO—R6,
—CONH—R6, —CON(R6)₂, —O—R6, —S—R6, carboxy, acetamido, cyano, nitro, halogen, hydroxy, trifluoromethyl, trifluoromethoxy, sulfamoyl, carbamoyl, or hydroxymethyl;
R5, if present, is halogen, C₁-C₁₀ alkyl, C₁-C₁₀ alkoxy, C₁-C₁₀ alkylamino, C₁-C₁₀ dialkylamino, benzyl, benzyloxy, hydroxymethyl, nitro, trifluoromethyl, trifluoromethoxy, trifluoromethylthio, N-hydroxyimino, cyano, carboxy, acetamido, hydroxy, sulfamoyl, or carbamoyl;
R6, if present, is C₁-C₁₀ alkyl, C₂-C₁₀ alkenyl, C₂-C₁₀ alkynyl, C₃-C₁₀ cycloalkyl, or C₅-C₁₀ cycloalkenyl; wherein any one of said alkyl, alkenyl, alkynyl, cycloalkyl, or cycloalkenyl is optionally substituted with aryl, heteroaryl, benzyl, or phenethyl; said aryl or heteroaryl being optionally substituted with one or more R5 independently.

35. A method for treating a neurological disorder, comprising:
administering to a patient in need of such treatment a therapeutically effective amount of a compound of Formula Xa: 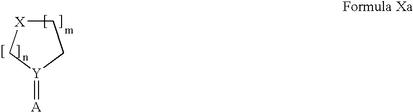
or of a pharmaceutically acceptable derivative thereof;
wherein X is CH₂, S, O, SO, SO₂, NH, or N(C₁-C₆ alkyl);
Y is N, CH, or C;
n is 1 or 2;
m is 0, 1, or 2;
the dashed bond symbol represents an optional bond;
and A is either:
an alpha-amino acyl group derived from an alpha-amino acid bearing a mono- or bicycloaliphatic side chain, said side chain being saturated or partially saturated, and optionally containing one or more heteroatoms;
or A is:
a beta-amino acyl group of the formula 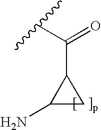
wherein p is 1-6, and the ring in said beta-amino acyl group is saturated or partially saturated, and optionally contains one or more heteroatoms;
wherein the 1′ carbonyl group in said alpha- or beta-amino acyl groups is optionally replaced by CH or CF.

36. A method for treating a neurological disorder, comprising:
administering to a patient in need of such treatment a therapeutically effective amount of a compound of Formula Xb: 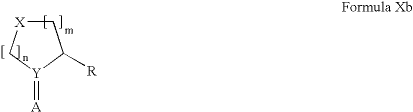
or of a pharmaceutically acceptable derivative thereof;
wherein X, Y, m, and n are as defined for Formula Xa of claim 35 above;
R is CN, C═C—R7, or CH═N—R8;
R7 is hydrogen, fluoro, nitro, C₁-C₆ alkyl, C₁-C₆ alkoxy, C₁-C₆ alkoxycarbonyl, or C₁-C₆ alkanoyl;
R8 is phenyl, hydroxy, C₁-C₆ alkoxy, —O—(CO)—(C₁-C₆ alkyl), or benzyloxy;
A is as defined for Formula Xa of claim 35 above, and in addition may be derived from any L-alpha-amino acid bearing a lipophilic side chain.

37. A method for treating a neurological disorder, comprising: administering to a patient in need of such treatment a therapeutically effective amount of a compound of Formula Xc: 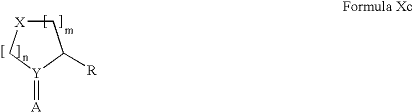
or of a pharmaceutically acceptable derivative thereof;
wherein X, Y, m, and n are as defined for Formula Xa of claim 35 above;
R is CHO or B(OH)₂;
A is a beta amino acyl group as defined for Formula Xa of claim 35 above.

38. A method for treating a neurological disorder, comprising:
administering to a patient in need of such treatment a therapeutically effective amount of a compound of Formula Xd: 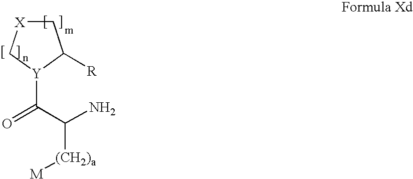
or of a pharmaceutically acceptable derivative thereof;
wherein X, Y, m, and n are as defined for Formula Xa of claim 35 above;
R is H, CN, C═C—R7, or CH═N—R8, wherein R7 and R8 are as defined for Formula Xb of claim 36 above;
a is 1-5;
M is:
—COO—(CH₂)_{b}—(R4)_{q}—R3,
—CONH—(CH₂)_{b}—R4)_{q}—R3,
—CONCH₃—(CH₂)_{b}—(R4)_{q}—R3,
—SO₂—NH—(CH₂)_{b}—(R4)_{q}—R3, or
—SO₂—NCH₃—(CH₂)_{b}—(R4)_{q}—R3;
wherein b is 0-12; q is 0-5;
R4 is Z—NH—(CH₂)_{c}— or NH—Z—(CH₂)_{c}—;
wherein c is 1-12; and
Z is CO, CH₂, or SO₂; and
R3 is
COOH,
—(COO)—(C₁-C₈ alkyl or fluoroalkyl),
—(COO)—(C₁-C₈ cycloalkyl),
—(COO)-aryl,
—(COO)-heteroaryl,
CONH₂,
CONHNH₂,
CONR5R6,
CONNR5R6,
PO₃H,
PO₃—(C₁-C₈ alkyl or fluoroalkyl),
PO₃—(C₁-C₈ cycloalkyl), PO₃-aryl,
PO₃-heteroaryl,
SO₃H,
SO₂NH₂,
SO₂NR5R6,
OH,
OR5,
NH₂,
NR5R6,
NHCOOR5,
NHSO₂NR5R6,
NHCOR5,
NHSO₂R5,
NH—CH(:NR5)NR5R6,
NHCONR5R6,
aryl, or heteroaryl, wherein said aryl or heteroaryl is mono- or bicyclic, the individual rings consisting of 5-6 members, and being optionally substituted with one or more substituents selected from the group consisting of F, Cl, I, Br, OH, OR5, NO₂, SO₃H, SO₂NH₃, SO₂NR5R6, NH₂, NR5R6, COOR5, CF₃, CN, CONH₂, CONR5R6, NHCOOR5, CH(:NR5)NR5R6, NH—CH(:NR5)NR5R6 and R5;
sugar, which is attached via an ether or a glycosidic bond;
CO-aminosugar which is attached via its amino group;
NHCO-aminosugar, or
NHCS-aminosugar;
wherein R5 and R6 are independently selected from H, C₁-C₈ straight or branched chain alkyl, C₁-C₈ straight or branched chain fluoroalkyl, C₃-C₈ cycloalkyl, and aryl, heteroaryl, or alkylheteroaryl of up to 11 atoms;
or wherein R5 and R6 together optionally form a 3-8-membered carbocyclic chain.

39. A method for treating a neurological disorder, comprising: administering to a patient in need of such treatment a therapeutically effective amount of a compound of Formula Xe:
Formula Xe 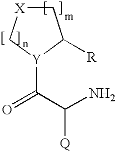
or of a pharmaceutically acceptable derivative thereof;
wherein X, Y, m, and n are as defined for Formula Xa of claim 35 above;
R is as defined for Formula Xd of claim 38 above;
Q is a group selected from 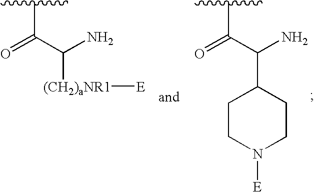
R1 is H or CH₃;
E is
—(CO)—(CH₂)_{b}—(R4)_{q}—R3,
—CH₂—(CH₂)_{b}—R4)_{q}—R3; or
—SO₂—(CH₂)_{b}—(R4)_{q}—R3;
wherein a, b, q, R3, and R4 are as defined for Formula Xd of claim 38 above.

40. A method for treating a neurological disorder, comprising: administering to a patient in need of such treatment a therapeutically effective amount of a compound of Formula Xf: 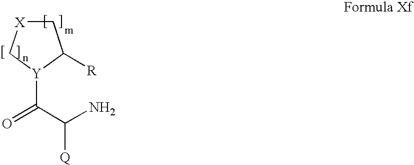
or of a pharmaceutically acceptable derivative thereof;
wherein X, Y, m, and n are as defined for Formula Xa above;
R is as defined for Formula Xd above;
Q is a group selected from 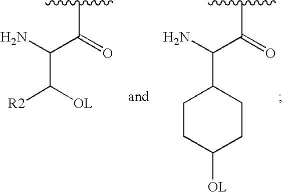
L is
—(CH₂)_{d}—(CO)ᵣ—(CH₂)_{b}—R4)_{q}—R3, or
—(CH₂)ₑ—NR1—(CH₂)_{b}—(R4)_{q}—R3;
R1 and R2 are independently H or CH₃;
r is 0 or 1;
d is 0-4;
e is 2-4; and
b, q, R3 and R4 are as defined for Formula Xd of claim 38 above.

41. A method for treating a neurological disorder, comprising:
administering to a patient in need of such treatment a therapeutically effective amount of a compound of Formula XI: 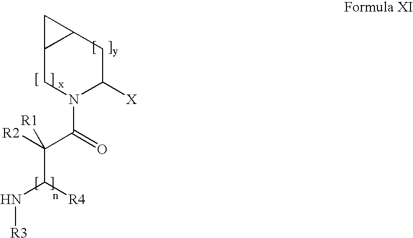
or of a pharmaceutically acceptable derivative thereof;
wherein x and y are independently 0 or 1, provided that only one of x and y can be 0;
n is 0 or 1;
X is H or CN;
R1, R2, R3, and R4 are independently selected from hydrogen, alkyl, alkenyl, alkynyl, cycloalkyl, cycloalkylalkyl, bicycloalkyl, tricycloalkyl, alkylcycloalkyl, hydroxyalkyl, hydroxyalkylcycloalkyl, hydroxycycloalkyl, hydroxybicycloalkyl, hydroxytricycloalkyl, bicycloalkylalkyl, alkylthioalkyl, arylalkylthioalkyl, cycloalkenyl, aryl, aralkyl, heteroaryl, heteroarylalkyl, cycloheteroalkyl or cycloheteroalkylalkyl; all optionally substituted through available carbon atoms with 1, 2, 3, 4 or 5 groups selected from hydrogen, halo, alkyl, polyhaloalkyl, alkoxy, haloalkoxy, polyhaloalkoxy, alkoxycarbonyl, alkenyl, alkynyl, cycloalkyl, cycloalkylalkyl, polycycloalkyl, heteroarylamino, arylamino, cycloheteroalkyl, cycloheteroalkylalkyl, hydroxy, hydroxyalkyl, nitro, cyano, amino, substituted amino, alkylamino, dialkylamino, thiol, alkylthio, alkylcarbonyl, acyl, alkoxycarbonyl, aminocarbonyl, alkynylaminocarbonyl, alkylaminocarbonyl, alkenylaminocarbonyl, alkylcarbonyloxy, alkylcarbonylamino, arylcarbonylamino, alkylsulfonylamino, alkylaminocarbonylamino, alkoxycarbonylamino, alkylsulfonyl, aminosulfinyl, aminosulfonyl, alkylsulfinyl, sulfonamido or sulfonyl;
and wherein R1 and R3 may optionally be taken together to form a group
—(CR5R6)ₘ— where m is 2 to 6, and R5 and R6 are the same or different and are independently selected from hydroxy, alkoxy, H, alkyl, alkenyl, alkynyl, cycloalkyl, halo, amino, substituted amino, cycloalkylalkyl, cycloalkenyl,
aryl, arylalkyl, heteroaryl, heteroarylalkyl, cycloheteroalkyl, cycloheteroalkylalkyl, alkylcarbonylamino, arylcarbonylamino, alkoxycarbonylamino, aryloxycarbonylamino, alkoxycarbonyl, aryloxycarbonyl, or alkylaminocarbonylamino,
or R1 and R4 may optionally be taken together to form —(CR7R8)ₚ— wherein p is 2 to 6, and R7 and R8 are the same or different and are independently selected from hydroxy, alkoxy, cyano, H, alkyl, alkenyl, alkynyl, cycloalkyl, cycloalkylalkyl, cycloalkenyl, halo, amino, substituted amino, aryl, arylalkyl, heteroaryl, heteroarylalkyl, cycloheteroalkyl, cycloheteroalkylalkyl, alkylcarbonylamino, arylcarbonylamino, alkoxycarbonylamino, aryloxycarbonylamino, alkoxycarbonyl, aryloxycarbonyl, or alkylaminocarbonylamino, or optionally R1 and R3 together with 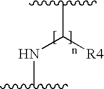
form a 5 to 7 membered ring containing a total of 2 to 4 heteroatoms selected from N, O, S, SO, or SO₂;
or optionally R1 and R3 together with 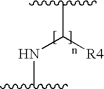
form a 4 to 8 membered cycloheteroalkyl ring wherein the cycloheteroalkyl ring has an optional aryl ring fused thereto or an optional 3 to 7 membered cycloalkyl ring fused thereto.

42. A method for treating a neurological disorder, comprising: administering to a patient in need of such treatment a therapeutically effective amount of a compound of Formula II of claim 26 above, Formula V, VI, VII, VIII, IX, Xa, Xb, Xc, Xd, Xe, Xf, or XI, wherein said neurological disorder is a neurodegenerative disorder; neuropathic disorder; neurovascular disorder;
traumatic injury of the brain, spinal cord, or peripheral nervous system;
demyelinating disease of the central or peripheral nervous system; metabolic or hereditary metabolic disorder of the central or peripheral nervous system; or
toxin-induced- or nutritionally related disorder of the central or peripheral nervous system.

43. The method of claim 42, wherein said neurodegenerative disorder is Parkinson's disease, Alzheimer's disease, amyotrophic lateral sclerosis (ALS), Huntington's disease, cerebellar ataxia, or multisystem atrophy.
